(19) United States
(12) Patent Application Publication
(10) Pub. No.: US 2022/0401566 A1
(43) Pub. Date: Dec. 22, 2022

(54) PEG-LIPID

(71) Applicant: ICOAT MEDICAL AB, Stockholm (SE)

(72) Inventors: Bo NILSSON, Uppsala (SE); Kristina NILSSON EKDAHL, Uppsala (SE); Marianne JENSEN WAERN, Knivsta (SE); Yuji TERAMURA, Tokyo (JP)

(21) Appl. No.: 17/776,644

(22) PCT Filed: Dec. 8, 2020
(86) PCT No.: PCT/SE2020/051177

§ 371 Date: May 13, 2022

(30) Foreign Application Priority Data
Jan. 15, 2020 (SE) 2050025-2

(51) Int. Cl. A61K 47/61; A61K 47/60; A61K 47/54; A61K 47/69; A61P 7/02

(52) U.S. Cl. CPC A61K 47/61; A61K 47/60; A61K 47/543; A61K 47/6911; A61P 7/02

(57) ABSTRACT

A PEG-lipid is produced by mixing a cation-PEG-lipid comprising at least one amino group with a sulfated glycosaminoglycan comprising at least one carbonyl group to form a Schiff base intermediate. A reducing agent is added to the Schiff base intermediate to form a sulfated glycosaminoglycan-PEG-lipid. The sulfated glycosaminoglycan-PEG-lipid can be used to biological tissue against thromboinflammation. Coating of biological tissue with the sulfated glycosaminoglycan-PEG-lipid can be done in a single step process and does not cause any significant cell aggregation.

### TECHNICAL FIELD

The present invention generally relates to poly(ethylene glycol) (PEG) lipids, and in particular to such PEG-lipids comprising sulfated glycosaminoglycans, and production and medical uses thereof.

### BACKGROUND

Although no severe side effects have been reported after cell transplantation of, for instance, islets of Langerhans, mesenchymal stem cells (MSCs) or hepatocytes, the bioincompatibility of these therapeutic cells has remained unresolved. The infusion of therapeutic cells into the human body is associated with a large loss of transplanted cells as the result of an immune reaction termed thromboinflammation or instant blood-mediated inflammatory reaction (IBMIR). Thromboinflammation, or IBMIR, is an innate immune attack triggered by the complement and coagulation systems that is followed by a rapid binding of platelets and infiltration of leukocytes into the clot, resulting in early loss of the transplanted cells. In addition, thromboinflammation reaction occurs in ischemia reperfusion injury (IRI) in solid organ transplantation, such as kidney and heart transplantation. This devastating reaction destroys tissues and organs after the transplantation, which reduces the graft survival.

Therefore, it is critical to protect the cell surfaces from this thromboinflammatory attack in order to achieve successful treatment and a high-level engraftment of the therapeutic cells and solid organ.

Some studies have shown that the thromboinflammation can be regulated via systemic administration of anticoagulants, such as the thrombin inhibitor, melagatran, low-molecular weight dextran sulfate, and/or complement inhibitors to prevent early unfavorable reactions. However, some of these techniques are difficult to apply in the clinical setting because of the associated increased risk of bleeding.

Heparan sulfate is expressed on endothelial cell surfaces and plays an important role in regulating coagulation as well as complement and platelet activation. Therefore, mimicking the endothelial surface by surface modification with heparin and heparin conjugates has been suggested as an approach in regulating the thromboinflammation that occurs in cell and organ transplantations [1-3]. However, surface modification with heparin and heparin conjugates requires several process steps; chemical modification of cell surface and reaction with heparins with washing processes required after each step.

Another problem associated with surface modification with heparin and heparin conjugates is cell aggregation after the reaction with heparin. The heparin molecules also cross-link between cells, thereby causing cell clumping.

There is therefore a need for compounds that can be used to protect biological tissue against thromboinflammation and that does not have shortcomings associated with prior art solutions.

### SUMMARY

It is a general objective to provide molecules useful in protecting biological tissue against thromboinflammation and that do not have at least some of the shortcoming associated with prior art solutions.

This and other objectives are met by the invention as defined herein.

The invention is defined in the independent claims. Further embodiments of the invention are defined in the dependent claims.

An aspect of the invention relates to a method of producing a poly(ethylene glycol) lipid (PEG-lipid). The method comprises mixing a cation-PEG-lipid comprising at least one amino group with a sulfated glycosaminoglycan comprising at least one carbonyl group, preferably at least one aldehyde group, to form a Schiff base intermediate. The method also comprises adding a reducing agent to the Schiff base intermediate to form a sulfated glycosaminoglycan-PEG-lipid.

Another aspect of the invention relates to a PEG-lipid comprising at least one sulfated glycosaminoglycan attached to the PEG-lipid via a bond formed between an amino group of a cation-PEG-lipid comprising at least one amino group and a carbonyl group of the at least one sulfated glycosaminoglycan comprising at least one carbonyl group to form a Schiff base intermediate that is reduced by a reducing agent.

Further aspects of the invention relate to a biological tissue comprising at least one such PEG-lipid anchored in cell membrane of the biological tissue and a liposome comprising at least one such PEG-lipid anchored in a lipid bilayer of the liposome.

Aspects of the invention also define a PEG-lipid according to the invention for use as a medicament, for use in treatment of thromboinflammation, for use in treatment of instant blood mediated reaction (IBMIR), for use in treatment of ischemia reperfusion injury (IRI), for use in treatment of stroke and for use in treatment of myocardial infarction.

Another aspect of the invention relates to an in vitro method of providing biological tissue with a sulfated glycosaminoglycan coating. The in vitro method comprises adding in vitro PEG-lipids according to the invention to the biological tissue to anchor the PEG-lipids in cell membranes of the biological tissue.

A further aspect of the invention defines an ex vivo method of treating an organ or a part of the organ.

The method comprises ex vivo infusing a solution comprising PEG-lipids according to the invention into a vascular system of the organ or the part of the organ. The method also comprises ex vivo incubating the solution comprising PEG-lipids according to the invention in the vascular system to enable coating of at least a portion of the endothelial lining of the vascular system with the PEG-lipids according to the invention.

The PEG-lipids of the present invention can be used to coat lipid membrane structures, such as cells and liposomes, by a single step procedure. Such a coating of the lipid membrane structures furthermore does not cause any significant aggregation or clumping of the cells or liposomes. The PEG-lipids of the present invention can thereby be used to protect biological tissue against thromboinflammation but without the shortcomings associated with prior art solutions.

### BRIEF DESCRIPTION OF THE DRAWINGS

The embodiments, together with further objects and advantages thereof, may best be understood by making reference to the following description taken together with the accompanying drawings, in which:

FIG. 1 is a schematic illustration of heparin-conjugated PEG-lipids (fHep-lipids). fHep-lipid: fHep-C-lipid, fHep-K1C-lipid, fHep-K2C-lipid, fHep-K4C-lipid, and fHep-K8C-lipid.

FIG. 2 schematically illustrates synthesis of fHep-lipid. (A) Mal-PEG-lipid was reacted with C, K1C, K2C, K4C, or K8C, followed by conjugation with fragmented heparin (fHep). (B) Unfractionated heparin (UFH) was fragmented into fragmented heparin (fHep). (C) fHep-KnC-lipid (n=0, 1, 2, 4, 8).

FIG. 3 is a diagram illustrating absorbance of fHep and heparin at 260 nm (N=3).

FIG. 4 are diagrams illustrating (A) molecular weight analysis by gel permeation chromatography (GPC) (N=9 for fragmented heparin (fHep), N=8 for unfractionated heparin) and (B) anti-factor Xa activity of fHep and unfractionated heparin (N=4).

FIG. 5 are diagrams illustrating (A) size and (B) zeta potential of fHep-lipids (N=3).

FIG. 6 illustrates a quartz crystal microbalance with dissipation monitoring (QCM-D) based analysis for antithrombin (AT) binding activity of fHep-lipid.

FIG. 7 is a diagram illustrating quantitative analysis for the binding amount of AT to fHep-lipid and cation-PEG-lipid (N=3).

FIG. 8 illustrates a QCM-D-based analysis for AT-binding activity of fHep(−)-lipid.

FIG. 9 are diagrams illustrating quantitative analysis for the binding amount of AT and bovine serum albumin (BSA) to fHep(−)-lipid (N=3).

FIG. 10 illustrates a QCM-D-based analysis for factor H-binding activity of fHep(−)-lipid.

FIG. 11 are diagrams illustrating (A) quantitative analysis for the binding amount of factor H and AT to fHep(−)-lipid and Mal-PEG-lipid (N=3) and (B) the calculated molar ratio of immobilized factor H to fHep(−)-lipids and Mal-PEG-lipid (N=3).

FIG. 12 is a diagram illustrating anti-factor Xa activity of fHep-lipid modified liposomes (N=3).

FIG. 13 is a diagram illustrating size of fHep-lipid modified liposomes (N=3).

FIG. 14 is a diagram illustrating polydispersity index (PDI) of fHep-lipid modified liposomes (N=3).

FIG. 15 is a diagram illustrating zeta potential of fHep-lipid modified liposomes (N=3).

FIG. 16 show fluorescence images of AT (Alexa488 labeled) on the surface of human red blood cells which were treated with fHep(−)-lipid, K1C-PEG-lipid and fHep.

FIG. 17 is a diagram illustrating quantitative analysis for the binding amount of AT (Alexa488 labeled) on the surface of red blood cells treated with fHep(−)-lipid, K1C-PEG-lipid and fHep by flow cytometry.

FIG. 18 is a diagram illustrating anti-factor Xa activity of fHep-lipid modified CCRF-CEM cells (*: p<0.05, N=3).

FIG. 19 illustrates influence on blood compatibility of hMSCs modification with fHep-lipid. (A) Confocal images of hMSCs treated with fHep-lipid and Alexa488-labeled AT. Here fHep-lipid is fHep-K1C(−)-lipid and fHep-K8C(−)-lipid. Scale bar: 40 μm (B) Quantitative analysis of AT-binding onto modified hMSCs by flow cytometry. Error bars indicate standard deviation (N=5). (C) Viability assay of modified hMSCs by trypan blue exclusion method. Error bars indicate standard deviation (N=5). (E)-(G) Loop model assay of modified hMSCs in human whole blood. Modified hMSCs were incubated in human whole blood (0.5 IU/mL UFH) with 1.0×10⁵ cells/mL for 2 hr at 37° C. Here, hMSCs were modified with fHep-KnC(−)-lipid (n=1 and 8), and K1C-PEG-lipid. PBS-added whole blood and non-treated hMSCs were used as a control. The figures show (D) relative platelet count and generation of (E) TAT, (F) C3a, and (G) sC5b-9. Error bars indicate standard deviation (N=6).

FIG. 20 illustrates loop model assay of modified hMSCs in human whole blood. Modified hMSCs were incubated in human whole blood (0.5 IU/mL UFH) with 1.0×10⁴ cells/mL for 2 hr at 37° C. Here, hMSCs were modified with fHep-KnC(−)-lipid (n=1 and 8), and K1C-PEG-lipid. PBS-added whole blood and non-treated hMSCs were used as a control. The figures show (A) relative platelet count and generation of (B) TAT, (C) C3a, and (D) sC5b-9. Error bars indicate standard deviation (N=6).

### DETAILED DESCRIPTION

The present invention generally relates to poly(ethylene glycol) (PEG) lipids, and in particular to such PEG-lipids comprising sulfated glycosaminoglycans, and production and medical uses thereof.

The PEG-lipids of the present invention are useful in surface modifications of cell and organ transplants to mimic the endothelial surface and thereby protect such cell and organ transplants against thromboinflammation. The PEG-lipids have several advantages as compared to prior art approaches using heparin and heparin conjugates. Firstly, the surface modification with the PEG-lipids of the present invention can be performed in a single step without the need for any chemical modification of the cell surface. This means that the surface modification process of cell or organ transplants with the PEG-lipids can be performed much easier as compared to the prior art requiring several process steps including chemical modification of the cell surface, which may cause adverse effects to the cells.

Secondly, the PEG-lipids of the invention do not cross-link when attached to cells. Thereby, the PEG-lipids are not marred by the shortcomings of the prior art causing cell clumping and aggregation after reaction with heparin or heparin conjugates.

The PEG-lipids of the invention are therefore useful in protecting biological tissue, including cell and organ transplants, against thromboinflammation.

An aspect of the invention relates to a method of producing a PEG-lipid. The method comprises mixing a cation-PEG-lipid comprising at least one amino group with a sulfated glycosaminoglycan comprising at least one carbonyl group, preferably at least one aldehyde group, to form a Schiff base intermediate.

The method also comprises adding a reducing agent to the Schiff base intermediate to form a sulfated glycosaminoglycan-PEG-lipid.

The glycosaminoglycan-PEG-lipid is formed by Schiff base chemistry involving nucleophilic addition forming a hemiaminal followed by a dehydration to generate a Schiff base intermediate. The starting material in this reaction is a cation-PEG-lipid comprising at least one amino group. This at least one amino group reacts with at least one carbonyl group, preferably at least one aldehyde group, of the sulfated glycosaminoglycan to form the Schiff base intermediate (C═N bond between the sulfated glycosaminoglycan and the cation-PEG-lipid) that is reduced by the addition of the reducing agent to form the sulfated glycosaminoglycan-PEG-lipid with the sulfated glycosaminoglycan attached to the PEG-lipid through a C—N bond.

Hence, the sulfated glycosaminoglycan is attached to the cation-PEG-lipid through a covalent bond, and in more detail a covalent bond between a C in a carbonyl group, preferably an aldehyde group, of the sulfated glycosaminoglycan and an N in an amino group of the cation-PEG-lipid, i.e., a C—N bond.

The cation-PEG-lipid comprising at least one amino group could be any PEG-lipid, including PEG-phospholipid, comprising at least one amino group.

A PEG-lipid may have the general structure of formula (II) with a corresponding PEG-phospholipid according to the general structure of formula (III), wherein R₁ and R₂ represent the lipid parts of the molecule.

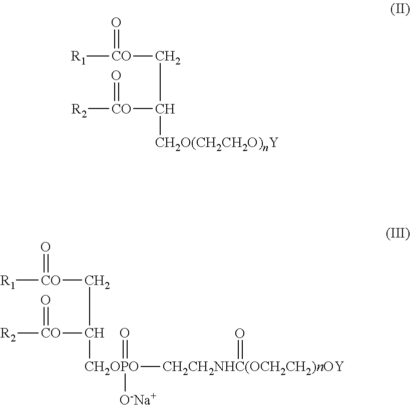

Y in formula (II) and (III) is, in an embodiment, selected from the group consisting of H, CH₃, maleimide and N-hydroxysuccinimide.

PEG-lipid as used herein comprises any conjugate between PEG and at least one lipid, including fatty acids, phospholipids, glycerolipids, glycerophospholipids, sphingolipids, sterols, prenols, saccharolipids, and polyketides. In a preferred embodiment, the PEG-lipid is selected to be able to be anchored in a lipid layer, such as in the cell membrane of a biological material. A currently preferred PEG-lipid is a PEG-phospholipid.

The at least one amino group is preferably introduced into the PEG-lipid to form the cation-PEG-lipid formed by reacting a maleimide-conjugated PEG-lipid with a cysteine peptide.

Hence, in an embodiment, the method comprises an additional step of mixing a maleimide-conjugated PEG-lipid with at least one cysteine peptide to form the cation-PEG-lipid comprising at least one amino group. In an embodiment, the at least one cysteine peptide can be at least one KₙC peptide, at least one CKₙ peptide or a combination thereof, wherein C is cysteine, K is lysine and n is zero or a positive integer equal to or smaller than 20, preferably equal to or smaller than 15, more preferably equal to or smaller than 10, such as 0, 1, 2, 3, 4, 5, 6, 7, 8, 9 or 10.

If n=0 in the KnC or CKₙ peptide, then the cation-PEG-lipid will comprise a single amino group. Each lysine in the KₙC or CKₙ peptide adds one amino group to the cation-PEG-lipid, which therefore comprises n+1 amino groups.

In an embodiment, the maleimide-conjugated PEG-lipid is formed by mixing α-N-hydroxysuccinimidyl-ω-maleimidyl PEG (NHS-PEG-Mal), triethylamine and 1,2-dipalmitoyl-sn-glycerol-3-phosphatidylethanolamine (DPPE) in dicholoromethane. The maleimide-conjugated PEG-lipid is then precipitated by adding diethyl ether to the mixture of NHS-PEG-Mal, triethylamine and DPPE in dicholormethane.

The sulfated glycosaminoglycan comprises at least one carbonyl group. A currently preferred carbonyl group is an aldehyde group (—CHO). However, the invention is not limited thereto but also encompasses sulfated glycosaminoglycans comprising at least one aldehyde group, at least one ketone (—C(═O)—), at least one carboxyl group (—C(═O)OH), at least one carboxylate ester group (—C(═O)O—) and/or at least one amide group (—C(═O)NR— or —C(═O)NH—). The sulfated glycosaminoglycan can comprise a single carbonyl group, such as a single aldehyde group, or multiple, i.e., at least two, carbonyl groups, such as multiple aldehyde groups.

The glycosaminoglycan (GAG) is a long linear polysaccharide comprising repeating disaccharide units, i.e., a plurality of disaccharide units. Most often the repeating unit comprises an amino sugar, e.g. N-acetylglucosamine or N-acetylgalactosamine, along with a uronic sugar, e.g., glucuronic acid or iduronic acid, or galactose. In an embodiment, the sulfated glycosaminoglycan is selected from the group consisting of a heparin, a heparan sulfate, a chondrotin sulfate, a dermatan sulfate, a keratin sulfate and hyaluronic acid.

A currently preferred sulfated glycosaminoglycan is a heparin comprising at least one carbonyl group, preferably heparin comprising at least one aldehyde group. In a particular embodiment, the sulfated glycosaminoglycan is fragmented heparin (fHep) comprising at least one carbonyl group, preferably fragmented heparin comprising at least one aldehyde group.

Such a fragmentation of heparin introduces a carbonyl group, preferably an aldehyde group, to the heparin molecule. Furthermore, the fragmentation reduces the length of the heparin chain and thereby the molecular weight as compared to unfractionated heparin (UFH).

In an embodiment, the fragmentation reaction comprises mixing an acidic solution and a sodium nitrite (NaNO₂) aqueous solution to form a mixed solution. The pH of the mixed solution is adjusted within an interval of from 2 up to 6, preferably from 3 up to 5, and more preferably 4. Heparin, preferably in the form of heparin sodium, is added to the mixed solution to form a heparin solution. The pH of the heparin solution is adjusted within an interval of from 6 to 8, preferably from 6.5 to 7.5 and more preferably to 7 to form the fragmented heparin comprising at least one carbonyl group, preferably at least one aldehyde group. The fragmentation reaction may optionally comprise dialyzing the fragmented heparin comprising at least one carbonyl group, preferably at least one aldehyde group, against water and lyophilizing the fragmented heparin comprising at least one carbonyl group, preferably at least one aldehyde group.

The acidic solution is preferably selected from a sulfuric acid (H₂SO₄) solution or an acetic acid (CH₃COOH) solution, preferably sulfuric acid (H₂SO₄) solution.

In an embodiment, adding the reducing agent comprises adding sodium cyanoboronhydride (NaBH₃CN) to the Schiff base intermediate to form the sulfated glycosaminoglycan-PEG-lipid. Hence, in a preferred embodiment, the reducing agent is sodium cyanoboronhydride. The embodiments are, however, no limited thereto. Other reducing agents than sodium cyanoboronhydride could alternatively, or in addition, be used including, for instance, sodium triacetoxyborohydride and sodium borohydride.

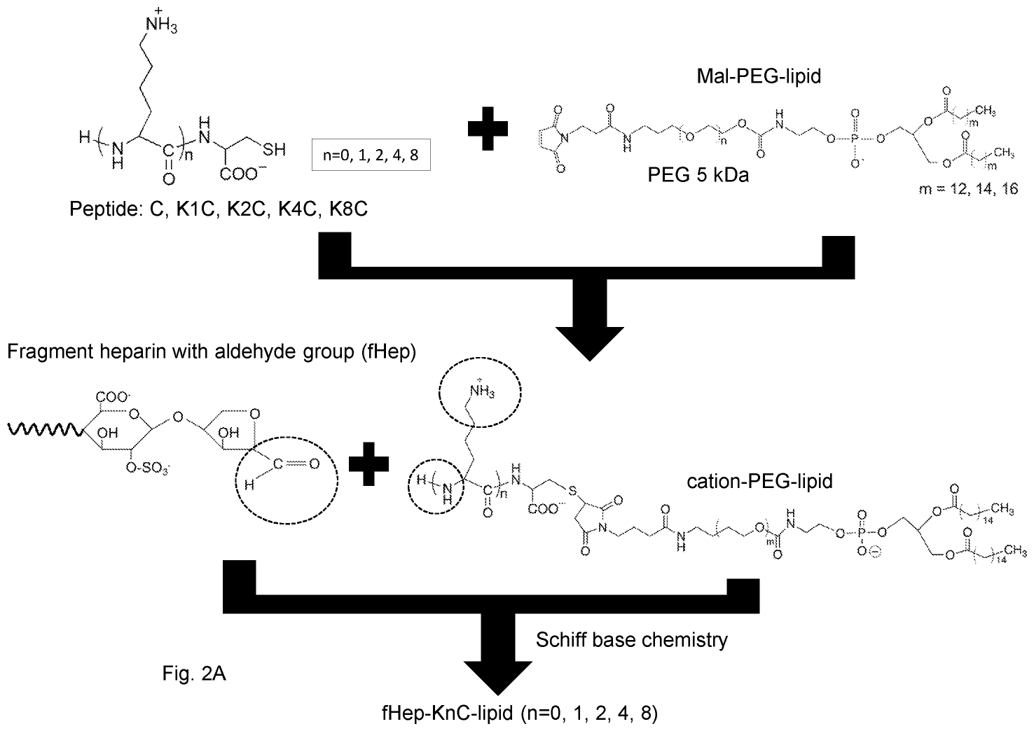

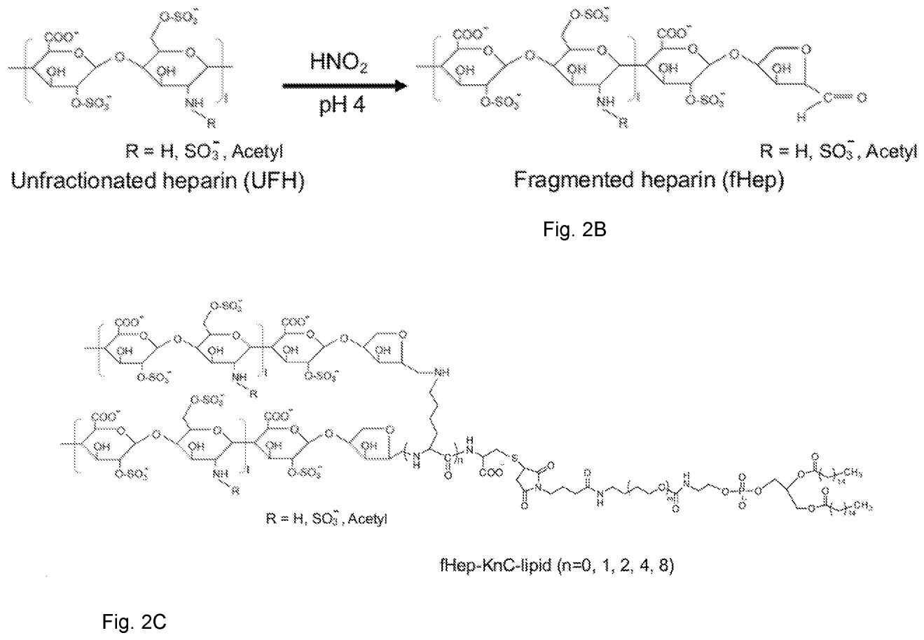

FIG. 2A schematically illustrates an example of synthesis of fHep-lipid. Mal-PEG-lipid was reacted with a C peptide (n=0), a K1C peptide (n=1), a K2C peptide (n=2), a K4C peptide (n=4), or a K8C peptide (n=8), followed by conjugation with fHep comprising an aldehyde group to the sulfated glycosaminoglycan-PEG-lipids fHep-KₙC-lipid. FIG. 2B illustrates fragmentation of unfractionated heparin (UHF) into fragmented heparin (fHep) and FIG. 2C illustrates an embodiment of a sulfated glycosaminoglycan-PEG-lipid.

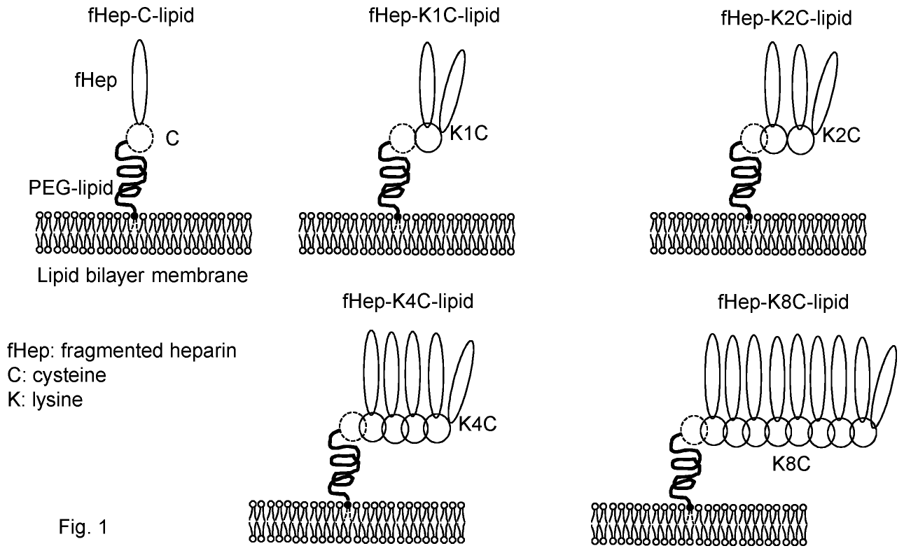

FIG. 1 schematically illustrates the sulfated glycosaminoglycan-PEG-lipids (fHep-KₙC-lipid) synthesized according to FIGS. 2A to 2C anchored into a lipid bilayer membrane. FIG. 1 also indicates the maximum number of fHep molecules per fHep-KₙC-lipid, i.e., n+1 fHep molecules.

In an embodiment, any unreacted amino groups in the sulfated glycosaminoglycan-PEG-lipid are converted into carboxylic groups.

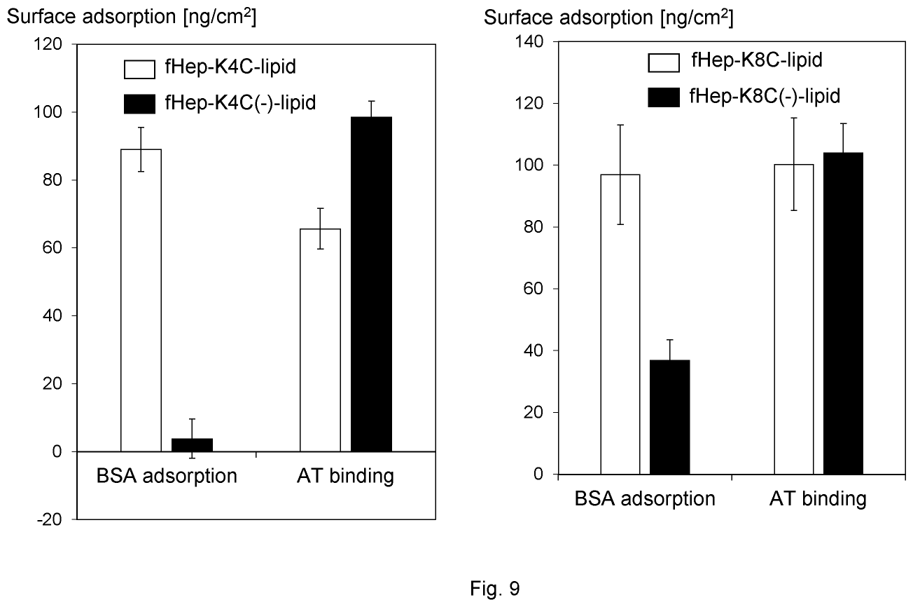

Carboxylic groups are generally less reactive than amino groups. Hence, converting unreacted amino groups in the sulfated glycosaminoglycan-PEG-lipid into carboxylic groups makes the sulfated glycosaminoglycan-PEG-lipid less cytotoxic and therefore less harmful to cells. In addition, the negative charges introduced by the carboxylic groups inhibit non-specific protein binding to a surface, at which the sulfated glycosaminoglycan-PEG-lipids are anchored, see FIG. 9.

In a particular embodiment, any such unreacted amino groups are converted into carboxylic groups by adding an anhydride to the sulfated glycosaminoglycan-PEG-lipid to convert any unreacted amino groups in the sulfated glycosaminoglycan-PEG-lipid into carboxylic groups.

Any anhydride could be used in the conversion of unreacted amino groups into carboxylic groups. Non-limiting, but illustrative, examples include succinic anhydride (SA), glutaric anhydride, diglycolic anhydride, and a combination thereof, preferably SA.

Another aspect of the invention relates to a PEG-lipid comprising at least one sulfated glycosaminoglycan.

The at least one sulfated glycosaminoglycan is attached to the PEG-lipid via bond formed between an amino group of a cation-PEG-lipid comprising at least one amino group and a carbonyl group of the at least one sulfated glycosaminoglycan comprising at least one carbonyl group to form a Schiff base intermediate that is reduced by a reducing agent.

Thus, according to the present invention, the sulfated glycosaminoglycan is attached to the PEG-lipid through a covalent bond, and in particular a covalent bond between a C in a carbonyl group, preferably an aldehyde group, of the sulfated glycosaminoglycan and an N in an amino group of the cation-PEG-lipid. This covalent bond between the carbon and nitrogen is a C—N bond.

In an embodiment, the PEG-lipid comprises a KₙC and/or CKₙ link interconnecting the at least one sulfated glycosaminoglycan and the PEG-lipid. In this embodiment, C is cysteine, K is lysine and n is zero or a positive integer equal to or smaller than 20. In an embodiment, n is selected within the interval of from 0 to 15, preferably within the interval of from 0 to 10, such as 0, 1, 2, 3, 4, 5, 6, 7, 8, 9 or 10.

In an embodiment, the sulfated glycosaminoglycan is attached to the PEG-lipid via a bond formed between an amino group of any lysine residue in the KₙC and/or CKₙ link or an N-terminal amine in the KₙC and/or CKₙ link and a carbonyl group, preferably an aldehyde group, of the at least one sulfated glycosaminoglycan comprising at least one carbonyl group, preferably at least one aldehyde group.

In an embodiment, the PEG-lipid part of the sulfated glycosaminoglycan-PEG-lipid has a formula (I)

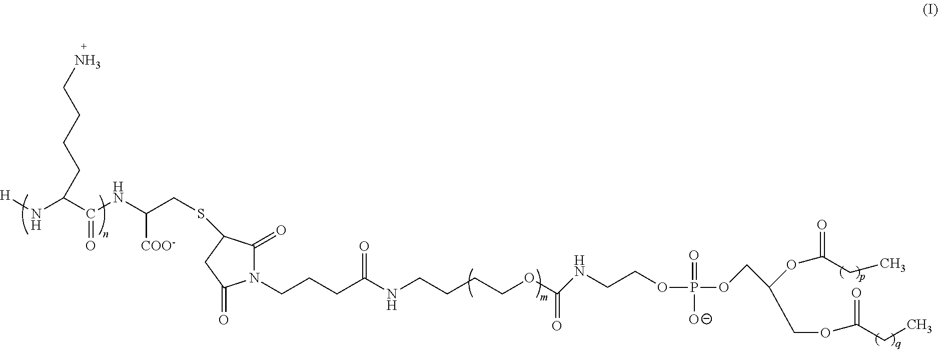

In formula (I), p, q are integers independently selected within the interval of from 10 up to 16, preferably p, q are independently 10, 12, 14 or 16, and more preferably p=q=14. m is selected so that the PEG chain has an average molecular weight selected within the range of from 1 kDa up to 40 kDa, preferably from 3 kDa up to 10 kDa and more preferably 5 kDa. Sulfated glycosaminoglycan molecules can then be attached to the PEG-lipid according to formula (I) at the N-terminal amine or at amino groups of the lysine residue(s).

Average molecular weight as defined herein indicates that individual PEG chains may have a molecular weight different from this average molecular weight but that the average molecular weight represents the mean molecular weight of the PEG chains. This further implies that there will be a natural distribution of molecular weights around this average molecular weight for a PEG chains.

In an embodiment, the sulfated glycosaminoglycan is fragmented heparin.

In an embodiment, the fragmented heparin has a weight average molecular weight (M_{w}) selected within the interval of from 2.5 kDa to 15 kDa, preferably within the interval of from 4 kDa to 10 kDa, such as within the interval of from 5 kDa to 10 kDa, and more preferably within the interval of from 5 kDa to 8 kDa or within an interval of from 7 kDa to 9 kDa.

In an embodiment, the sulfated glycosaminoglycan-PEG-lipid does not comprise any unreacted or free amino groups. In a particular embodiment, any unreacted or free amino groups in the sulfated glycosaminoglycan-PEG-lipid are converted into carboxylic groups.

Unreacted or free amino groups as referred to herein relate to any N-terminal amine and optional amino groups in any lysine residues in the PEG-lipid, such as illustrated in formula (I), that is not bound to any sulfated glycosaminoglycan molecule.

In an embodiment, the sulfated glycosaminoglycan-PEG-lipid is obtainable or obtained by the method as disclosed herein.

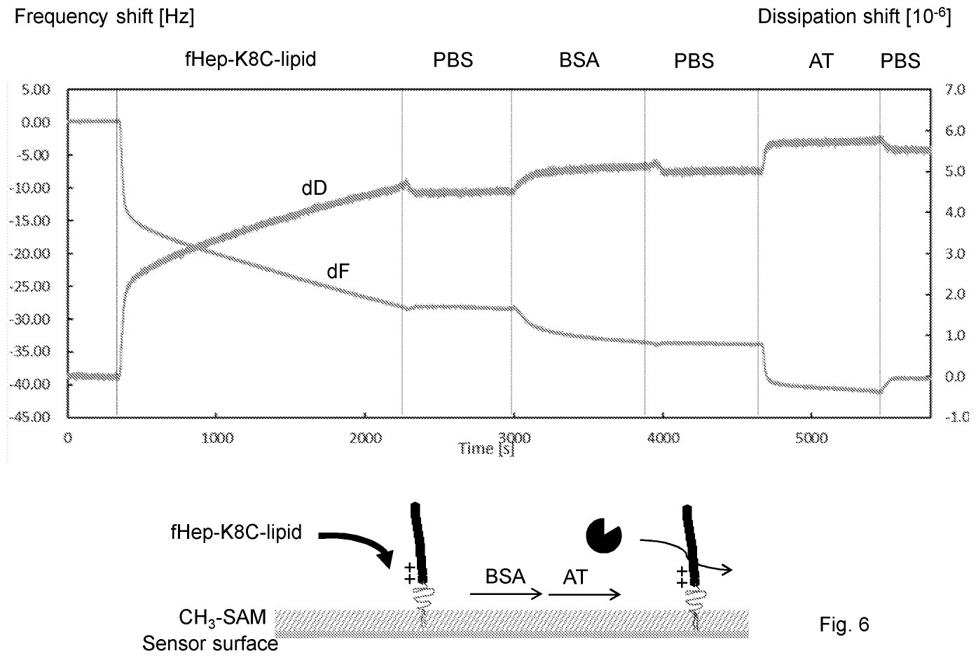

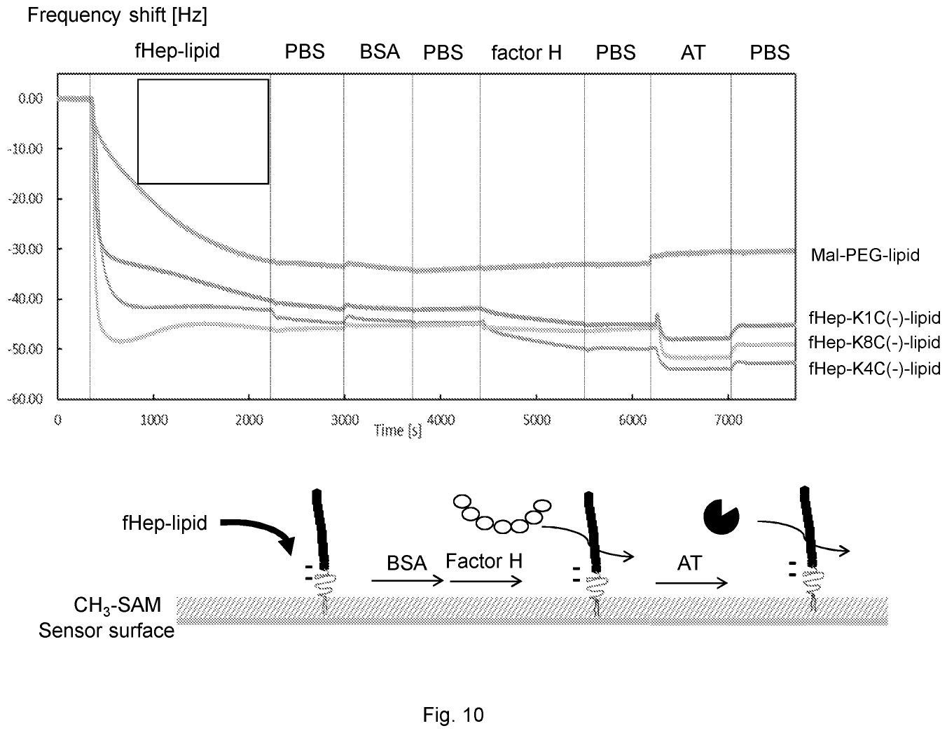

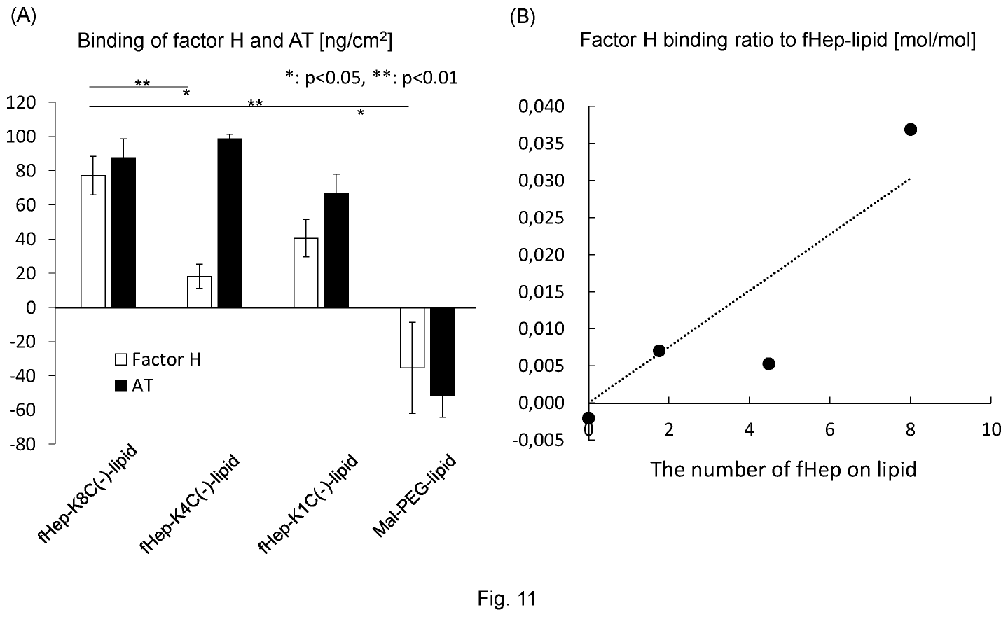

The sulfated glycosaminoglycan-PEG-lipids of the invention have affinity for antithrombin (AT), see FIGS. 6-10, 11A, and Factor H, see FIGS. 10, 11A and 11B.

AT is a protein molecule that inactivates several enzymes of the coagulation system. Its activity is increased manyfold by the anticoagulant drug heparin, which enhances the binding of AT to Factor IIa (thrombin) and Factor Xa (FXa). This means that the sulfated glycosaminoglycan-PEG-lipids of the invention have anti-FXa activity by being able to bind to AT and thereby have coagulation inhibiting effect.

Factor H is a member of the regulators of complement activation family and is a complement control protein. Its principal function is to regulate the alternative pathway of the complement system, ensuring that the complement system is directed towards pathogens or other dangerous material and does not damage host tissue. Factor H regulates complement activation on self cells and surfaces by possessing both cofactor activity for the Factor I mediated C3b cleavage, and decay accelerating activity against the alternative pathway C3-convertase, C3bBb. Factor H exerts its protective action on self cells and self surfaces but not on the surfaces of bacteria or viruses. This is thought to be the result of Factor H having the ability to adopt conformations with lower or higher activities as a cofactor for C3 cleavage or decay accelerating activity. The lower activity conformation is the predominant form in solution and is sufficient to control fluid phase amplification. The more active conformation is thought to be induced when Factor H binds to glycosaminoglycans and/or sialic acids that are generally present on host cells but not, normally, on pathogen surfaces ensuring that self surfaces are protected whilst complement proceeds unabated on foreign surfaces.

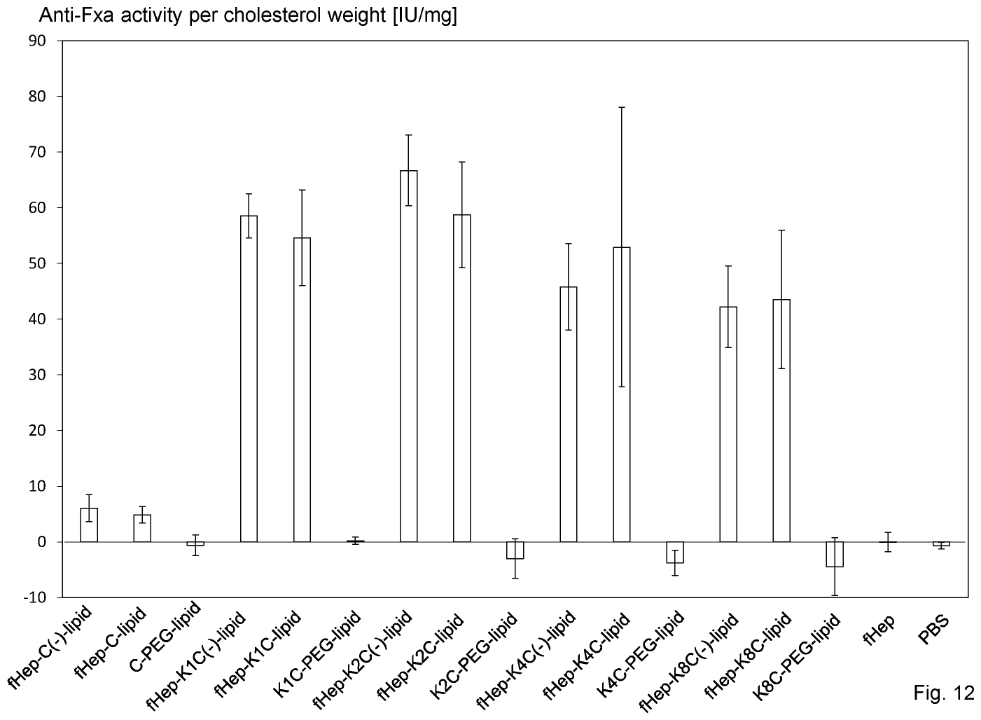

Hence, cell surfaces comprising anchored sulfated glycosaminoglycan-PEG-lipids of the present invention have the capability to attract and bind AT and Factor H and thereby protect the cell surfaces from thromboinflammation. The sulfated glycosaminoglycan-PEG-lipids of the invention have this biological effect even when attached to a lipid bilayer membrane, such as a cell surface or a liposome, see FIGS. 12, 17 and 18.

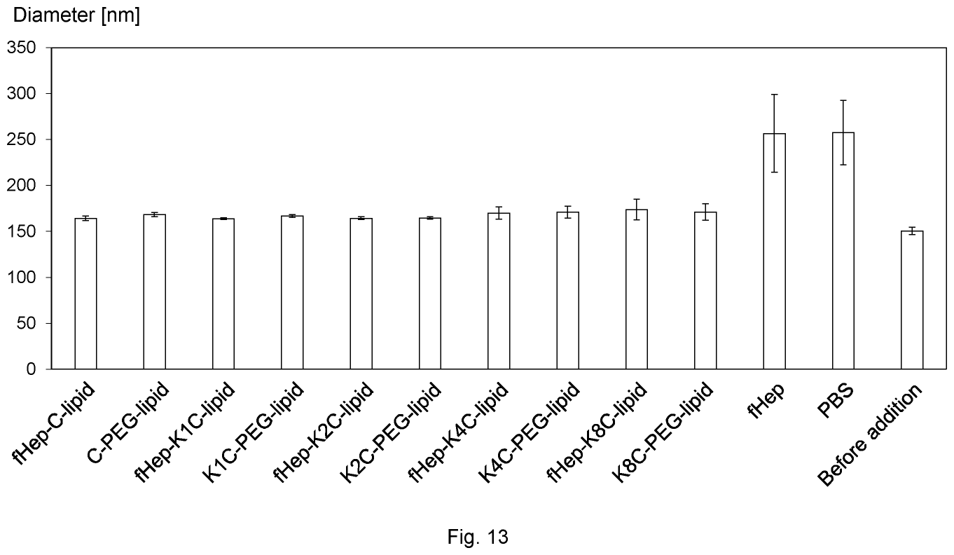

Experimental data as presented herein further shows that modifying lipid bilayer membranes with sulfated glycosaminoglycan-PEG-lipids of the present invention does not cause any aggregation or cell clumping, see FIG. 13, which is common when modifying cell surfaces with heparin according to the prior art.

The invention also relates to a lipid layer, preferably a lipid bilayer, comprising at least one sulfated glycosaminoglycan-PEG-lipid of the present invention. In such a case, the sulfated glycosaminoglycan-PEG-lipids are attached to or anchored into the lipid layer through the PEG-lipid group as indicated in FIG. 1. For instance, the invention relates to a liposome comprising at least one PEG-lipid according to the invention anchored in a lipid bilayer of the liposome.

A further aspect of the invention relates to a biological tissue comprising at least one PEG-lipid according to the present invention anchored in cell membrane of the biological tissue.

The biological tissue could be individual cells or multiple cells, such as stem cells, including mesenchymal stem cells (MSCs) and embryonic stem cells (ESCs); hepatocytes; endothelial cells; beta cells (insulin producing cells) and erythrocytes as illustrative, but non-limiting, examples. The biological tissue may alternatively be clusters of cells, such as islet of Langerhans. The biological tissue may also be in the form of a tissue or organ, or a part thereof, such as kidney, heart, pancreas, liver, lung, uterus, urinary bladder, thymus, intestine and spleen. In a particular embodiment, at least a portion of the vascular system, and optionally the parenchyma, of the tissue or organ, or the part thereof, may be coated with the at least one PEG lipid according to the present invention.

Another aspect of the invention relates to a PEG-lipid according to the invention for use as a medicament.

Further aspects of the invention relate to a PEG-lipid according to the invention for use in treatment of thromboinflammation, for use in treatment of instant blood mediated reaction (IBMIR), for use in treatment of ischemia reperfusion injury (IRI), for use in treatment of stroke and/or for use in treatment of myocardial infarction.

Related aspects of the invention define the use of a PEG-lipid according to the invention for the manufacture of a medicament for the treatment of thromboinflammation, IBMIR, IRI, stroke and/or myocardial infarction.

The PEG-lipids of the present invention may be administered to a subject in need thereof by systemic administration or local administration. Non-limiting examples of systemic administration routes include intravenous administration and subcutaneous administration. Local administration includes injection of the PEG-lipids of the present invention locally into a target organ or tissue in the subject.

The PEG-lipids of the present invention are preferably administered in the form of a PEG-lipid solution.

The solution comprising the PEG-lipid molecules could, for instance, be saline, an aqueous buffer solution or an organ preservation solution. Illustrative, but non-limiting, examples of aqueous buffer solutions that could be used include phosphate-buffered saline (PBS) and a citrate solution.

Another aspect of the invention relates to an in vitro method of providing biological tissue with a sulfated glycosaminoglycan coating. The in vitro method comprises adding in vitro PEG-lipids according to the invention to the biological tissue to anchor the PEG-lipids in cell membranes of the biological tissue.

An aspect of the invention relates to an ex vivo method of treating an organ or a part of an organ. The method comprises ex vivo infusing a solution comprising PEG-lipids according to the invention into a vascular system and, optionally into a parenchyma, of the organ or the part of the organ. The method also comprises ex vivo incubating the solution comprising PEG-lipids according to the invention in the vascular system, and optionally the parenchyma, to enable coating at least a portion of the endothelial lining of the vascular system, and preferably of the parenchyma, with the PEG-lipids according to the invention.

In an embodiment, the ex vivo incubating step comprises ex vivo incubating the solution comprising PEG-lipids according to the invention in the vascular system, and optionally the parenchyma, to enable coating at least a portion of the endothelial lining of the vascular system, and preferably of the parenchyma, with the PEG-lipids according to the invention while keeping the organ or the part of the organ submerged in an organ preservation solution, preferably an organ preservation solution comprising PEG-lipids according to the invention.

Thus, the ex vivo method comprises introducing PEG-lipids into the vascular system of the organ or a part of the organ and therein allow the PEG-lipid molecules to interact with and bind to the cell membranes of the endothelium and the parenchyma. FIG. 1 schematically illustrates this principle with the PEG-lipid molecules hydrophobically interacting with the lipid bilayer membrane to thereby anchor or attach the PEG-lipid molecules in the cell membrane through the phospholipid group.

The interaction between the PEG-lipid molecules with the lipid bilayer membrane of the endothelium and optionally of the parenchyma, such as renal parenchyma in the case of a kidney, is preferably taking place ex vivo while the organ or the part of the organ is submersed or submerged in an organ preservation solution, preferably an organ preservation solution comprising PEG-lipid molecules.

In a particular embodiment, the organ or the part of the organ is first ex vivo infused with the solution comprising PEG-lipid molecules into the vascular system and, optionally into the parenchyma, of the organ or the part of the organ. This ex vivo infusion is advantageously taking place as early as possible following explanting and removing the organ or the part of the organ from the donor body. The perfused organ or part of the organ is then submerged in the organ preservation solution, preferably comprising PEG-lipids, and kept therein, preferably at reduced temperature such as about 4° C.

In another particular embodiment, the organ or the part of the organ is first submerged into the organ preservation solution, preferably comprising PEG-lipid molecules, and then the solution comprising PEG-lipid molecules is ex vivo infused into the vascular system, and optionally into the parenchyma, of the organ or the part of the organ. This ex vivo infusion can be performed while keeping the organ or the part of the organ submerged in the organ preservation solution, preferably comprising PEG-lipid molecules. Alternatively, the organ or the part of the organ is temporarily removed from the organ preservation solution to perform the ex vivo infusion and is then put back into the organ preservation solution, preferably comprising PEG-lipid molecules.

In an embodiment, the method also comprises ex vivo infusing an organ preservation solution into the vascular system to flush away non-bound PEG-lipid molecules from the vascular system. Hence, non-bound PEG-lipid molecules are preferably washed away in one or multiple, i.e., at least two, wash steps using an organ preservation solution.

In an embodiment, ex vivo infusing the solution comprising PEG-lipid molecules comprises ex vivo clamping one of an artery and a vein of the vascular system. This embodiment also comprises ex vivo infusing the solution comprising PEG-lipid molecules into the other of the artery and the vein and ex vivo clamping the other of the artery and the vein.

In another embodiment, the solution with PEG-lipid molecules is infused into an artery (or vein) of the vascular system of the organ or the part of the organ until the solution appears at a vein (or artery) of the organ or the part of the organ. This confirms that the solution with PEG-lipid molecules has filled the vascular system. At that point, the artery and vein are clamped.

The solution comprising PEG-lipid molecules can be added either through a vein or through an artery. In a particular embodiment, the solution is infused into an artery. In such a particular embodiment, the optional, initial clamping is then preferably done of a vein of the vascular system.

The solution comprising PEG-lipid molecules is preferably ex vivo incubated in the vascular system for a period of time from 10 minutes up to 48 hours to enable the PEG-lipid molecules to hydrophobically interact with the cell membranes of the endothelium and thereby coat at least a portion of the vascular system of the organ or the part of the organ. The ex vivo incubation is preferably performed from 20 minutes up to 36 hours and more preferably from 30 minutes up to 24 hours, such as from 30 minutes up to 12 hours, up to 8 hours, up to 4 hours or up to 1 hour.

The amount of solution comprising PEG-lipid molecules infused into the vascular system depends on the type of the organ and the size of the organ (adult vs. child). Generally, the volume of the solution should be sufficient to fill the vascular system of the organ. In most practical applications, from 5 mL up to 250 mL of the solution comprising PEG-lipid molecules is ex vivo infused into the vascular system. In a preferred embodiment, from 5 mL up to 100 mL and preferably from 5 mL up to 50 mL solution comprising PEG-lipid molecules is ex vivo infused into the vascular system.

In an embodiment, the solution comprises from 0.25 mg/mL up to 25 mg/mL PEG-lipid molecules. In a preferred embodiment, the solution comprises from 0.25 mg/mL up to 10 mg/mL, preferably from 0.25 mg/mL up to 5 mg/mL, such as 2 mg/mL PEG-lipid molecules.

The above described concentrations of PEG-lipid molecules can also be used for the organ preservation solution comprising PEG-lipid molecules.

According to the invention, the solution comprising PEG-lipid molecules is ex vivo incubated in the vascular system while keeping the organ or the part of the organ submersed or submerged in an organ preservation solution, preferably comprising PEG-lipid molecules. Additionally, the organ or the part of the organ is preferably also kept in a temperature above 0° C. but below 8° C., preferably above 0° C. but equal to or below 6° C., and more preferably above 0° C. but equal to or below 4° C.

In this embodiment, the organ or the part of the organ is submerged in the organ preservation solution, preferably comprising PEG-lipid molecules, during the incubation time when the PEG-lipid molecules are allowed to interact with and bind to the cell membrane of the endothelium in the vascular system.

The organ or the part of the organ is preferably also kept cold, i.e., at a temperature close to but above 0° C. It has been shown that the theoretical perfect temperature for organ preservation is 4° C.-8° C. While higher temperatures lead to hypoxic injury of the organ because the metabolism is not decreased efficiently, lower temperatures than 4° C. increase the risk of cold injury with protein denaturation.

Currently, the gold standard for donor organ preservation in clinical organ transplantation uses three plastic bags and an ice box. The first plastic bag includes the organ itself immersed in an organ preservation solution. This first plastic bag is put in a second plastic bag filled with saline, and then these two plastic bacs are put in a third plastic bag filled with saline, which is then put in the ice box.

More advanced organ preservation devices for keeping organs in a temperature controlled environment are available and could be used, such as the Sherpa Pak™ transport systems from Paragonix Technologies, Inc. Waves from Waters Medical Systems, LifePort transporters from Organ Recovery systems, etc.

The solution comprising the PEG-lipid molecules could be saline, an aqueous buffer solution or an organ preservation solution.

Illustrative, but non-limiting, examples of aqueous buffer solutions that could be used include PBS and a citrate solution.

The organ preservation solution that could be used to infuse the PEG-lipid molecules and/or wash the vascular system of the organ or the part of the organ prior to or following ex vivo infusing PEG-lipid molecules and/or in which the organ or the part of the organ may be submerged can be selected from known organ preservation solutions. Illustrative, but non-limiting, examples of such organ preservation solutions include a histidine-tryptophan-ketoglutarate (HTK) solution, a citrate solution, a University of Wisconsin (UW) solution, a Collins solution, a Celsior solution, a Kyoto University solution and an Institut Georges Lopez-1 (IGL-1) solution.

The subject is preferably a human subject. The invention may, however, also be used in veterinary applications in which the subject is a non-human subject, such as a non-human mammal including, but not limited to, cat, dog, horse, cow, rabbit, pig, sheep, goat and guinea pig.

Further aspects of the invention relates to a method for treating, inhibiting or preventing thromboinflammation, IBMIR, IRI, stroke and/or myocardial infarction in a subject. The method comprises administering PEG-lipids according to the present invention to a subject in need thereof. In another embodiment, the method comprises the previously described method steps of ex vivo infusing a solution comprising PEG-lipids according to the present invention into a vascular system of the organ graft and ex vivo incubating the solution comprising PEG-lipid molecules in the vascular system to enable coating of at least a portion of the endothelial lining of the vascular system with the PEG-lipid molecules, optionally, but preferably, while keeping the organ graft submerged in an organ preservation solution preferably comprising PEG-lipid molecules.

The PEG-lipids according to the present invention enables a local protection against thromboinflammation by mimicking glycocalyx of normal endothelial cell surface. This approach can also avoid the risk of bleeding because the coating of endothelial cell surface in target organ requires small amounts of regulators compared to the systemic administration.

In an embodiment, the PEG-lipids of the present invention comprises heparin, which has similar functions to heparan sulfate proteoglycan (HS). Since heparin can interact with many regulators as same as HS, fHep-lipid coating obtained using the PEG-lipids of the present invention can regulate complex biological reactions during IRI, so that it can be easily applied for clinical trial.

Various methods of the heparin coating have been already reported in the art. A layer-by-layer coating of heparin together with soluble complement receptor 1 (sCR1) has been applied on mouse islet [7]. However, since the use of recombinant sCR1 is not practical and the procedures are complicated, the approach cannot be applicable to endothelial coating in kidney. Also, cationic avidin has used for the heparin coating of islets via electrostatic interaction [8]. However, it is difficult to use this method for clinical setting due to the strong antigenicity of avidin. Heparin-binding peptides have used for the immobilization of heparin by using PEG-lipid onto cellular surface [6, 9]. However, this coating procedure still needs several tedious processes, which makes it more difficult to coat endothelial surface of solid organs with heparin.

### EXAMPLES

The present Examples show the production and characterization of heparin-conjugated PEG-lipids (fHep-lipid), which can coat lipid membrane structures, such as cells and liposome, by a single-step process.

### Reagents and Materials

The following reagents and materials were used in the Examples:

Heparin sodium (UFH, FUJIFILM Wako Pure Chemical Corporation, Osaka, Japan)
Sulfuric acid (H₂SO₄, FUJIFILM Wako Pure Chemical Corporation)
Sodium nitrite (NaNO₂, FUJIFILM Wako Pure Chemical Corporation)
5 M Sodium hydroxide (NaOH, FUJIFILM Wako Pure Chemical Corporation)
Dialysis membrane (Spectra/Por, MWCO: 3.5-5 kDa, Repligen Corpolation, Waltham, Mass., USA)
Sodium cyanoborohydride (NaCNBH₃, Sigma-Aldrich Chemical Co., St. Louis, Mo., USA)

### D-PBS(−) (FUJIFILM Wako Pure Chemical Corporation)

### Biophen Heparin (AT+) (COSMO BIO Co., LTD., Tokyo, Japan)

### Dextran (M_{w}: 1080 Da, 9890 Da, 43500 Da, 123600 Da, Sigma-Aldrich Chemical Co.)

Sodium chloride (NaCl, FUJIFILM Wako Pure Chemical Corporation)
Distilled water (FUJIFILM Wako Pure Chemical Corporation)
Dimethyl sulfoxide (DMSO, FUJIFILM Wako Pure Chemical Corporation)
α-N-hydroxysuccinimidyl-ω-maleimidyl poly(ethylene glycol) (NHS-PEG-Mal, M_{w}: 5000 Da, NOF Corporation, Tokyo, Japan)
Triethylamine (Sigma Aldrich Co, St. Louis, Mo.)
1,2-dipalmitoyl-sn-glycerol-3-phosphatidylethanolamine (DPPE, NOF Corporation)

### Dichloromethane (Sigma Aldrich Chemical Co)

1,6-diphenyl-1,3,5-heatriene (DPH, Sigma Aldrich Chemical Co)

### L-cysteine (C, M_{W}=121.16 Da, FUJIFILM Wako Pure Chemical Corporation)

### Lysine-Cysteine (K1C, M_{w}=249.335 Da, BEX Co., Ltd., Tokyo, Japan)

### Lysine-Lysine-Cysteine (K2C, M_{W}=377.51 Da, BEX Co., Ltd.)

### Lysine-Lysine-Lysine-Lysine-Cysteine (K4C, M_{w}=633.85 Da, GenScript, Tokyo, Japan)

### Lysine-Lysine-Lysine-Lysine Lysine-Lysine-Lysine-Lysine-Cysteine (K8C, M_{w}=1146.54 Da, GenScript)

### Fluorescamine (FUJIFILM Wako Pure Chemical Corporation)

### Glycine (FUJIFILM Wako Pure Chemical Corporation)

Antithrombin (AT, KENKETU NONTHRON 500 for injection, Takeda Pharmaceutical Company limited, Osaka, Japan)

### 1-Dodecanethiol (FUJIFILM Wako Pure Chemical Corporation)

Fetal bovine serum albumin (BSA, Sigma-Aldrich Chemical Co.)

### Cholesterol (FUJIFILM Wako Pure Chemical Corporation)

Dipalmitoyl phosphatidylcholine (DPPC, MC-6060, NOF Corporation)
Poly(2-methacryloyloxyethyl phosphorylcholine-co-n-butyl methacrylate) (MPC polymer, composed of 3:7 ratio of 2-methacryloyloxyethyl phosphorylcholine (MPC) and n-butyl methacrylate (BMA) domain, NOF Corporation, Tokyo, Japan)
Polyoxyethylene sorbitan monolaurate (TWEEN® 20, TOKYO Chemical Industry Co., Ltd, Tokyo, Japan)
3,3′,5,5′-tetramethylbenzidine (TMB, ready-to-use solution, TOKYO Chemical Industry Co., Ltd, Tokyo, Japan)

### Ethanol (99.5%, FUJIFILM Wako Pure Chemical Corporation)

Citric acid monohydrate (CAM, FUJIFILM Wako Pure Chemical Corporation)
Sodium dodecyl sulfate (SDS, FUJIFILM Wako Pure Chemical Corporation)
Cholesterol quantification kit (T-Cho E, FUJIFILM Wako Pure Chemical Corporation)
Dioxane (dehydrated) (KANTO CHEMICAL)
Succinic anhydride (SA, FUJIFILM Wako Pure Chemical Corporation)
Trypan blue (Thermo Fisher Scientific, Waltham, Mass., USA)

### Dulbecco's Modified Eagle Medium (DMEM, Thermo Fisher Scientific, Waltham, Mass., USA)

### Trypsin-EDTA (0.25%, Thermo Fisher Scientific, Waltham, Mass., USA)

### CCRF-CEM (American Type Culture Collection, ATCC, Manassas, Va., USA)

Human mesenchymal stem cells (hMSCs, Lonza, Morristown, N.J., USA)
Horse radish peroxidase (HRP)-conjugated streptavidin (GE healthcare, Chicago, Ill., USA)
RPMI 1640 medium (Invitrogen, Carlsbad, Calif., USA)

### Fatal Bovine Serum (FBS, Thermo Fisher Scientific)

Penicillin-Streptomycin, Liquid (P/S Penicillin: 5000 IU/mL, Streptomycin: 5000 μg/mL in 100 mL of
0.85% NaCl aqueous solution, Thermo Fisher Scientific)
Alexa Fluor™ 488 Antibody Labeling Kit (including sodium bicarbonate and Alexa Fluor™ 488 carboxylic acid, tetrafluorophenyl (TFP) ester in the kit, Thermo Fisher Scientific)
Vacuum blood collection tube (EDTA-2Na treated, TERUMO Corporation, Tokyo, Japan)
Ethylenediaminetetraacetic acid solution, (EDTA, 0.5 M, pH 8.0, Invitrogen)
Factor H (purified from human blood)

### Equipment

The following equipment was used in the Examples:

pH meter (LAQUA, HORIBA, Kyoto, Japan)

### Nanodrop-1000 (Thermo Fisher Scientific)

### Nanodrop-3300 (Thermo Fisher Scientific)

Quartz crystal microbalance with energy dissipation (QCM, qsense, Biolin scientific, Gothenburg,

### Sweden)

Gel permeation chromatography (GPC, LC-2000Plus series, JASCO, Tokyo, Japan)

### Zetasizer Nano ZS (Malvern Instruments Co., Ltd., Worcestershire, UK)

Plate reader (AD200, Beckman Coulter, Miami, Fla., USA)
Cell counter (countess, Invitrogen)

### Extruder (Avanti Polar Lipids, Inc., Avanti Polar Lipids, Inc., Birmingham, Ala., USA)

### Centrifuge (MX301, TOMY SEIKO Co, Ltd., Tokyo, Japan)

Centrifuge (Force mini SBC 140-115, BM EQUIPMENT Co., LTD, Tokyo, Japan)
Confocal laser scanning microscopy (CLSM, LSM880, Carl Zeiss, Jena, Germany)
Flow cytometer (FCM, BD LSR II, BD Biosciences, San Jose, Calif., USA)

### Example 1—Synthesis and Characterization of Fragmented Heparin (fHep)

Synthesis of Fragmented Heparin

Sulfuric acid (H₂SO₄) solution (1 M) and sodium nitrite (NaNO₂) aqueous solution (7 M) were mixed and the pH of the mixed solution was adjusted to 4. A solution of heparin sodium (unfractionated heparin (UFH), 20 mg/mL in water, 3 mL) was mixed with the mixed solution of H₂SO₄ and NaNO₂ (11 mL) for 15 min at room temperature (RT, ˜20-25° C.). Then, pH of the solution was adjusted to 7 by adding 1 M NaOH aqueous solution (approximately 4 mL). After the reactant was dialyzed against MilliQ water using dialysis membrane (3.5-5 kDa, Spectra/Por) for 1 day, the solution was lyophilized to obtain fragmented heparin (fHep). The yield was 40%

UV Spectrum

A solution of fHep (10 mg/mL, in PBS) was measured by UV-vis spectrophotometer (Nanodrop 1000, Thermo Fisher Scientific, Waltham, Mass., USA) in order to check the aldehyde group of fHep.

The Measurement of Molecular Weight of fHep Using GPC

The molecular weight of UFH and fHep was measured by GPC. The column was Shodex SB803HQ (Showa Denko, Tokyo, Japan). The eluent was 0.1 M of NaCl aqueous solution. The flow speed was 0.5 mL/min, and the temperature of the column oven was 25° C. As the standard reagent, dextran (M_{w}: 1080 Da, 9890 Da, 43500 Da, 123600 Da) (Sigma-Aldrich Chemical Co., St. Louis, Mo., USA) was used.

FXa Assay to Examine the Heparin Activity

The anti-factor Xa activity of synthesized fHep was evaluated using a FXa activity assay kit (Biophen Heparin (AT+), COSMO BIO Co., LTD.). The concentration of fHep was 0.01 mg/mL (in PBS) while that of UFH as a standard was 2, 1, 0.5 IU/mL.

Results

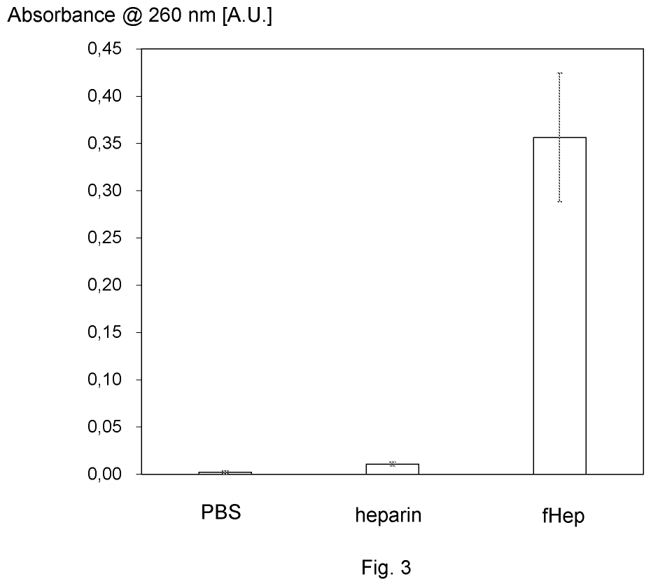

Since the aldehyde group has an absorbance at 260 nm, the fHep solution has absorbance at that wavelength (FIG. 3). As is shown in FIG. 3, there was no absorbance for original UFH. The results showed that fHep comprises an aldehyde group.

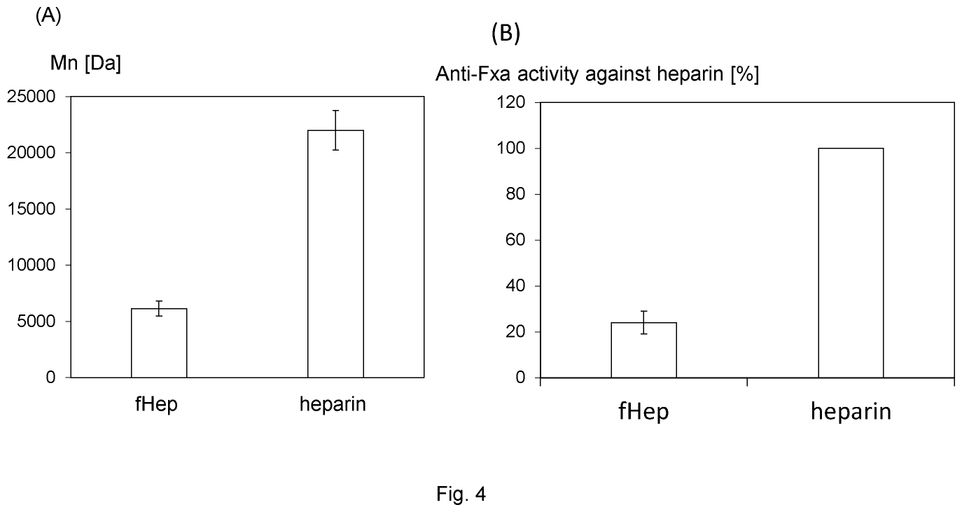

The number average molecular weight (Mₙ) of fHep and heparin was calculated by GPC using dextran standards (FIG. 4A). fHep had Mₙ=6.1 kDa, while UFH had Mₙ=22 kDa. The results showed that fHep was a fragmentation of heparin. The activity of fHep was measured by Factor Xa activity assay (FIG. 4B). The activity of fHep was approximately 24% of the activity of the original UFH.

### Example 2—Synthesis and Evaluation of Cation-PEG-Lipid and fHep-Lipid

Synthesis of Mal-PEG-lipid

The synthesis of Mal-PEG-lipid was performed as previously disclosed [4]. Briefly, α-N-hydroxysuccinimidyl-ω-maleimidyl poly(ethylene glycol) (NHS-PEG-Mal, M_{w}: 5000 Da, 200 mg), triethylamine (50 μL) and 1,2-dipalmitoyl-sn-glycerol-3-phosphatidylethanolamine (DPPE, 20 mg) were dissolved in dichloromethane and stirred for 48 h at RT. Precipitation with diethyl ether yielded Mal-PEG(5k)-lipid as a white powder (yield: 80%).

Synthesis of Cation-PEG-Lipid

In order to introduce at least one amine group at the end of the PEG chain, we conjugated C, K1C, K2C, K4C, and K8C to Mal-PEG-lipid where each lysine residue contains one amino group. C, K4C and K8C were dissolved in PBS, and K1C and K2C were dissolved in DMSO at a concentration of 10 mg/mL (stock solution). Each stock solution (10 mg/mL, 21 μL for C, 55 μL for K1C, 83 μL for K2C, 117 μL for K4C, or 217 μL for K8C) was mixed with Mal-PEG(5k)-lipid (10 mg/mL, 1000 μL, in PBS). Each resultant solution was rotated at RT for 24 h. The following cation-PEG-lipids were produced; C-PEG-lipid, K1C-PEG-lipid, K2C-PEG-lipid, K4C-PEG-lipid, and K8C-PEG-lipid, denoted KₙC-PEG-lipid (n: number of lysine residues) herein.

Synthesis and Functional Evaluation of fHep-Lipid

Each cation-PEG-lipid (1 mL, 10 mg/mL, in PBS) was mixed with fHep (15, 30, 45, 70, and 120 mg for C-, K1C-, K2C-, K4C-, and K8C-PEG-lipid, respectively), followed by addition of NaCNBH₃ solution (6, 13, 18, 30, and 49 μL for C-, K1C-, K2C-, K4C-, and K8C-PEG-lipid, respectively, 6.4 M, in PBS). The mixed solutions were stirred at RT for 3 days (for K8C-PEG-lipid and K4C-PEG-lipid) or 7 days (for K2C-PEG-lipid, K1C-PEG-lipid and C-PEG-lipid) to obtain the following fHep-lipids: fHep-C-lipid, fHep-K1C-lipid, fHep-K2C-lipid, fHep-K4C-lipid, and fHep-K8C-lipid.

After the reaction, succinic anhydride (SA) was added to change unreacted amine groups of fHep-lipids to carboxylic groups. Each fHep-lipid (1 mL, 10 mg/mL, in PBS) was mixed with SA solution (33, 64, 94, 151, and 252 μL for fHep-C—, fHep-K1C-, fHep-K2C-, fHep-K4C-, and fHep-K8C-lipid respectively, 0.5 M in dioxane) and stirred at room temperature for 24 h. Then, the resulting solution was lyophilized and purified by GPC (spin column, Pierce™ Polyacrylamide Spin Desalting Columns, 7K MWCO, 0.7 mL, Thermo Fisher Scientific) to obtain fHep(−)-lipids: fHep-C(−)-lipid, fHep-K1C(−)-lipid, fHep-K2C(−)-lipid, fHep-K4C(−)-lipid, and fHep-K8C(−)-lipid.

Determination of the Diameter and Surface Charge of fHep-Lipid

The diameter, polydispersity index (PDI) and zeta-potential (surface charge) of each cation-PEG-lipid (0.5 mg/mL in PBS), fHep-lipid (0.5 mg/mL in PBS) and fHep (4 mg/mL in PBS) were evaluated by dynamic light scattering using Zetasizer Nano ZS (Malvern Instruments Co., Ltd., Worcestershire, U.K).

Determination of Critical Micelle Concentration (CMC) for fHep-Lipid

DPH was used for measurement of CMC of fHep-lipid. fHep-lipid, cation-PEG-lipid and Mal-PEG-lipid (1 mL, 1.0×10⁻¹−1.0×10⁻⁷ mg/mL, in PBS) and DPH solution (2 μL, 30 μM, in THF) were mixed and incubated for 1 hr at 37° C. Then, the fluorescence intensity of the resultant solution was measured using fluorophotometer (FP-6600, JASCO, Ex: 357 nm, Em: 430 nm).

Determination of the Concentration of Amine Groups Using Fluorescamine

Each cation-PEG-lipid and fHep-lipid was diluted with PBS (0.5 mg/mL) and fluorescamine was dissolved in DMSO at a concentration of 3 mg/mL. Each cation-PEG-lipid solution (9 μL) or fHep-lipid (9 μL) was mixed with the fluorescamine solution (3 μL) for 15 min at RT and the absorbance (at 481 nm) of each resultant solution was measured by Nanodrop-3300 (Thermo Fisher Scientific, Waltham, Mass., USA). The same experiment was performed using C, K1C, K2C, K4C, and K8C solution with the same concentration. Glycine was used for the calibration curve to determine the amine group concentration.

Results

The molecular design of fHep-lipid is shown in FIG. 1. Multiple fragmented heparins can be conjugated to each PEG-lipid molecule (FIG. 2A). Heparin was chemically modified to obtain fragmented heparin having an aldehyde group at the end (fHep, FIG. 2B). Then, fHep was conjugated to cationic NH₂-PEG-lipid (cation-PEG-lipid) by Shiff base chemistry (FIGS. 2A, 2C). In order to introduce amine groups, C, K1C, K2C, K4C and K8C were used, which were conjugated to Mal-PEG-lipid. The following cation-PEG-lipids were produced: C-PEG-lipid (one amine group), K1C-PEG-lipid (two amine groups), K2C-PEG-lipid (three amine groups), K4C-PEG-lipid (five amine groups), K8C-PEG-lipid (nine amine groups).

fHep was conjugated to each cation-PEG-lipid through Shiff base chemistry between an aldehyde group and an amine group, followed by reduction with NaCNBH₃. By measuring both unreacted amine groups of the fHep-lipids and amine groups of the cation-PEG-lipids by using fluorescamine, the number of conjugated fHep to cation-PEG-lipid was calculated. The percentage of reacted amine group was calculated as 89%, 90%, 91%, 88%, and 61% for fHep-K8C-lipid, fHep-K4C-lipid, fHep-K2C-lipid, fHep-K1C-lipid and fHep-C-lipid, respectively as listed in Table 1 below. Then, the number of conjugated fHep to per PEG-lipid was 8.0, 4.5, 2.7, 1.8, and 0.6 for fHep-K8C-lipid, fHep-K4C-lipid, fHep-K2C-lipid, fHep-K1C-lipid and fHep-C-lipid, respectively.

TABLE 1
Number of conjugated fHep per PEG-lipid

| | Theoretical | | Calculated | |
|---|---|---|---|---|
| | number of | | number of | |
| | fHep per | Measured | fHep per | Calculated |
| | PEG-lipid | NH₂ (%) | PEG-lipid | MW (kDa) |
| fHep-C-lipid | 1 | 61 | 0.6 | 10 |
| fHep-K1C-lipid | 2 | 88 | 1.8 | 17 |
| fHep-K2C-lipid | 3 | 91 | 2.7 | 23 |
| fHep-K4C-lipid | 5 | 90 | 4.5 | 34 |
| fHep-K8C-lipid | 9 | 89 | 8.0 | 56 |

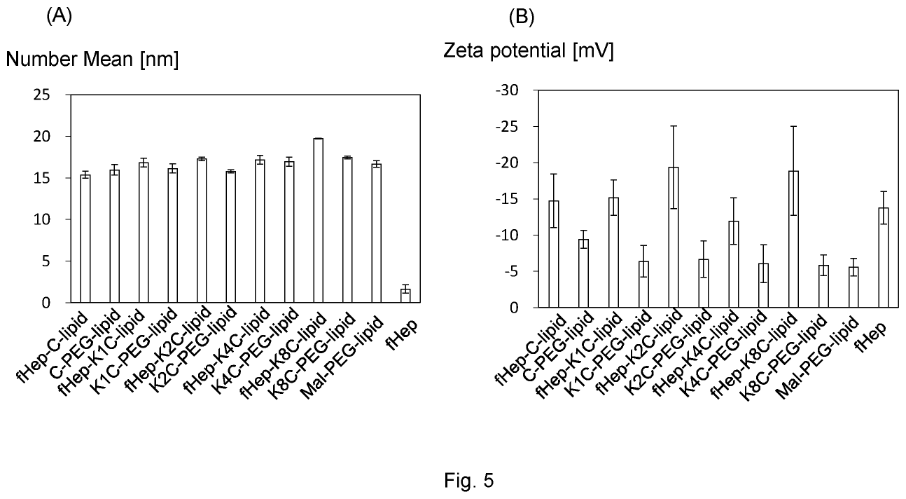

The micelle size of each fHep-lipid was determined by DLS (FIG. 5A). All fHep-lipids showed between 15 nm and 20 nm, while fHep showed around 2 nm. In addition, the zeta potential of each fHep-lipid was more negative than that of each cation-PEG-lipid (FIG. 5B). These results indicated that fHep was conjugated to PEG-lipid.

We also measured the CMC of each fHep-lipid using DPH. The CMC was 0.9, 1.1, 1.1, 1.0, 0.6, 1.1, 1.1, 1.0, 1.0, 0.7 and 1.1 μM for fHep-C-lipid, fHep-K1C-lipid, fHep-K2C-lipid, fHep-K4C-lipid, fHep-K8C-lipid, C-PEG-lipid, K1C-PEG-lipid, K2C-PEG-lipid, K4C-PEG-lipid, K8C-PEG-lipid and Mal-PEG-lipid respectively, indicating that fHep-lipid is amphiphilic and actually could form micelles.

### Example 3—Functional Evaluation of fHep-Lipid by QCM-D

The function of the fHep-lipids was evaluated by quartz crystal microbalance with energy dissipation (QCM-D, Q-sense, Gothenburg, Sweden). The binding capacity of antithrombin (AT) against each fHep-lipid and fHep(−)-PEG-lipid was quantified by QCM-D. After the QCM gold sensor chip was cleaned by oxygen plasma treatment (300 W, 100 mL/min gas flow, PR500; Yamato Scientific Co., Ltd., Tokyo, Japan), the sensor chip was immersed in 1-dodecanethiol solution (1.25 mM, in EtOH) for 24 hr to form hydrophobic self-assembled monolayer (CH₃—SAM). After intensive wash with ethanol and water, the sensor chip was set into the QCM-D chamber. A solution of each fHep-lipid (0.1 mg/mL in PBS) was flowed into the chamber for 30 min, then, BSA solution (1 mg/mL, in PBS) was flowed for 10 min for a blocking treatment. Finally, AT solution (0.1 mg/mL, in PBS) was flowed into the chamber for 10 min. PBS was flowed for 2 min for washing before each sample solution was flowed. The adsorption of each material was calculated from the resonance frequency change (Δf at the 7^{th} overtone) using the Sauerbrey equation [5].

In addition, the binding of Factor H to the fHep(−)-lipids (fHep-K1C(−)-lipid, fHep-K4C(−)-lipid and fHep-K8C(−)-lipid) or Mal-PEG-lipid (as control) was studied with QCM-D. A solution of each fHep(−)-lipid or Mal-PEG-lipid (0.1 mg/mL in PBS) was flowed into the chamber for 30 min, and BSA solution (1 mg/mL, in PBS) was flowed for 10 min for a blocking treatment. Then, Factor H solution (50 μg/mL, in PBS) was flowed into the chamber for 15 min. PBS was flowed for 2 min for the washing before each sample solution was flowed. After that, AT solution (0.1 mg/mL, in PBS) was flowed into the chamber for 10 min. PBS was flowed for 10 min for washing before each sample solution was flowed. The adsorption of each material was calculated from the resonance frequency change (Δf at the 7^{th} overtone) using the Sauerbrey equation [5].

Results

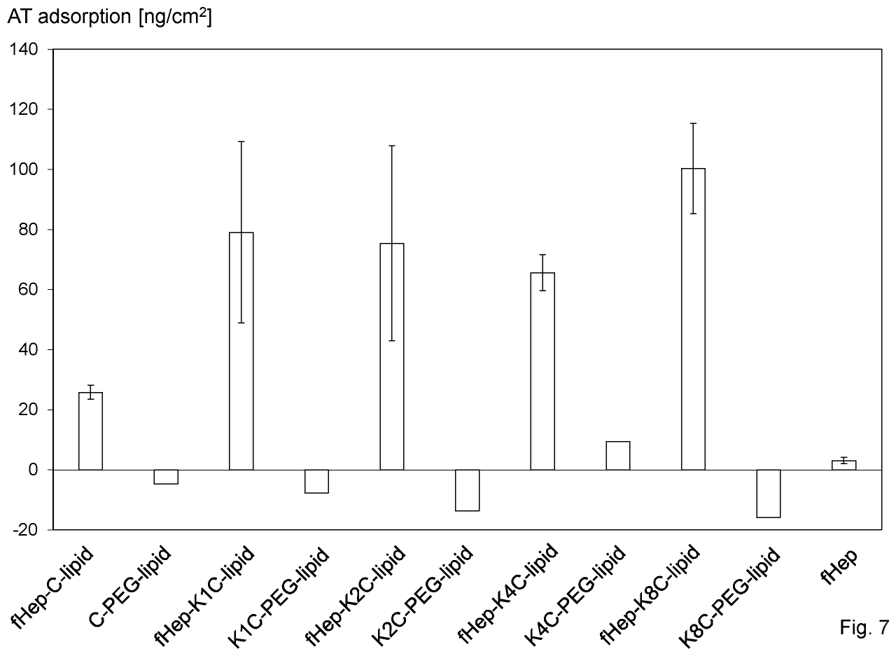

The binding ability of AT to the fHep-lipids was evaluated by QCM-D. FIG. 6 shows a representative QCM-D profile of interaction between fHep-K8C-lipid and AT. After blocking treatment with BSA, we could see the binding of AT to fHep-K8C-lipid on the surface. FIG. 7 summarizes the data of AT-binding amount for each fHep-lipid and cation-PEG-lipid. Here fHep was also added as a control. For all fHep-lipids, we could see the AT-binding, while there was no binding of AT to cation-PEG-lipid and fHep.

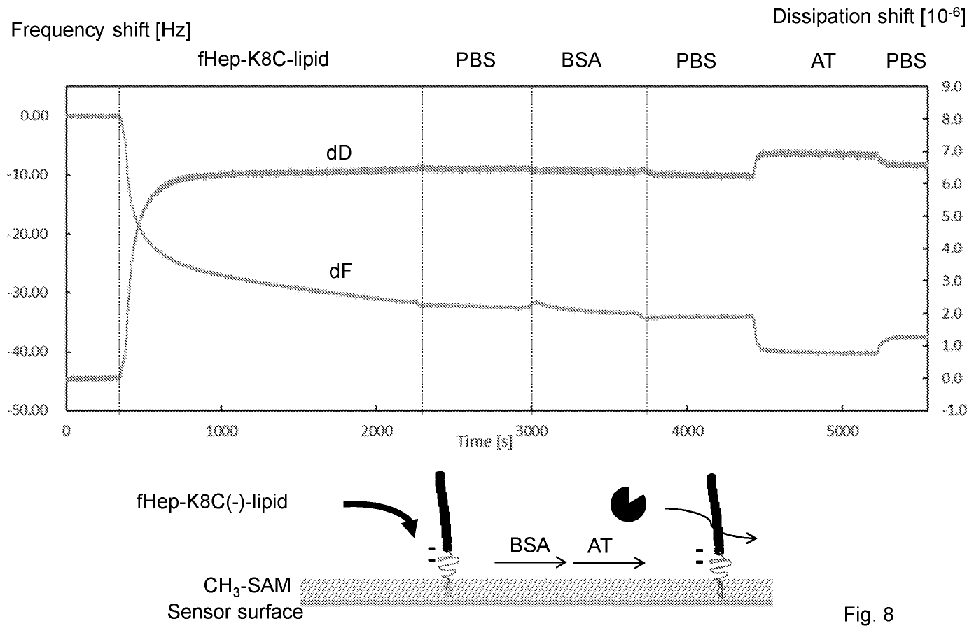

We also examined the AT-binding ability of fHep-lipids, which were treated with succinic anhydride (SA), i.e., fHep(−)-lipids. Since there are unreacted amine groups on fHep-lipids, SA was used to change them to carboxylic groups, which are less cytotoxic. FIG. 8 shows a representative QCM-D profile of interaction between fHep-K8C(−)-lipid and AT. When we added BSA for blocking treatment, we could see less binding of BSA, and only see the binding of AT (FIG. 9). Similar results were obtained when fHep-K4C(−)-lipid was used. These results showed that negative charge on fHep(−)-lipids inhibited non-specific binding of BSA.

We also examined the binding ability of Factor H to fHep(−)-lipids by QCM-D. FIG. 10 shows representative QCM-D profiles of interaction with Factor H. After blocking treatment with BSA, we could see the binding of Factor H onto fHep(−)-lipid whereas no binding was detected onto the control, Mal-PEG-lipid. FIGS. 11A and 11B summarize the quantitative analyses of Factor H-binding amount to each fHep(−)-lipid and Mal-PEG-lipid. For all fHep(−)-lipids, there was binding of Factor H, while there was no binding of Factor H to Mal-PEG-lipid. The number of Factor H per fHep(−)-lipid molecule was highest number when fHep-K8C(−)-lipid was used compared to fHep-K1C(−)-lipid and fHep-K4C(−)-lipid. This result suggests that highly packed fHep of fHep-K8C(−)-lipid has the highest affinity for Factor H, which is important for regulation of complement activation via recruiting Factor H.

### Example 4—Functional Evaluation of fHep-Lipid by FXa Activity Assay

The function of fHep-lipid was evaluated by FXa activity assay. Here we evaluated the binding capacity of antithrombin (AT) against each fHep-lipid, which was incorporated into liposomes.

Liposomes were prepared by dipalmitoyl phosphatidylcholine (DPPC) and cholesterol (1:1 by molar ratio). A cholesterol solution (530 μL, 10 mg/mL in ethanol) and DPPC solution (1 mL, 10 mg/mL in ethanol) were mixed and evaporated using a rotary evaporator to form a lipid film, followed by dried in vacuum for 24 hr. Then, PBS (1 mL) was added and vigorously stirred by a magnetic stir bar for 1 hr at RT. The resultant lipid suspension was extruded into membrane filters ($1000, 400, 200 and 100 nm) using an extruder (Avanti Polar Lipids, Birmingham, Ala., USA). The lipid suspension was passed through each filter 21 times.

To incorporate fHep-lipid into liposome surface, a solution of fHep-lipid was mixed with the liposome suspension. The liposome suspension (500 μL, 1 mg/mL in preparation, in PBS) was centrifuged (TOMY MX301, 20,000 g, 70 min, 4° C.), and then a fHep-lipid solution (50 μL, 0.5 mg/mL in PBS) was mixed with the liposome pellet. After incubation at RT for 10 min, the suspension was washed with PBS (450 μL) by centrifugation (20,000 g, 70 min, 4° C.) once. Finally, fHep-lipid-modified liposomes were obtained. The concentration of cholesterol in the liposomes was measured by an assay kit (T-Cho E, FUJIFILM Wako Pure Chemical Corporation). The FXa activity of the liposomes was evaluated using assay kit (Biophen Heparin (AT+), COSMO BIO Co., LTD).

FXa Activity Assay

Liposome suspension (15 μL, in PBS) was mixed with human AT (15 μL) in a 96 well-plate. Bovine FXa (75 μL) was added into each well and incubated at RT for 120 sec. Then, after coloring reagent (75 μL) was mixed for 90 sec, citric acid aqueous solution (100 μL, 20 mg/mL) was added. After each supernatant was collected by centrifugation (20,000 g, 70 min, 4° C.), the absorbance (at 405 nm) was measured.

The cholesterol of liposome was measured by mixing the liposome suspension (60 μL in PBS) with SDS (2 μL, 15 mg/mL, in PBS) at RT for 30 min for the solubilization. Then, cholesterol concentration was determined according to the company's instruction.

Results

Anti-FXa activity of fHep-lipid modified liposomes was evaluated (FIG. 12). As control groups, each cation-PEG-lipid modified liposomes and fHep-treated liposomes were used for the assay. The anti-FXa activity was normalized by liposome concentration. All fHep-lipid modified liposomes showed higher anti-FXa activity than the control groups. In addition, similar results were obtained when fHep(−)-lipid modified liposomes were used (FIG. 12). These results showed that the surface of liposomes can be modified with fHep-lipid and fHep(−)-lipid and that such modified liposomes have anti-FXa activity.

### Example 5—Characterization of Treated Liposomes

The surface of liposome was modified with each fHep-lipid (fHep-C-lipid, fHep-K1C-lipid, fHep-K2C-lipid, fHep-K4C-lipid, and fHep-K8C-lipid) or cation-PEG-lipid (C-PEG-lipid, K1C-PEG-lipid, K2C-PEG-lipid, K4C-PEG-lipid, and K8C-PEG-lipid) as described in Example 4. Also, fHep and PBS were used as control groups.

A solution of fHep-lipid (0.5 mg/mL in PBS) or cation-PEG-lipid (0.5 mg/mL in PBS) was mixed with liposome pellet after centrifugation (TOMY MX301, 20,000 g, 70 min, 4° C.). After the incubation at RT for 10 min, the liposomes were washed with PBS (450 μL) by centrifugation (20,000 g, 70 min, 4° C.) once. Finally, the fHep-lipid-modified liposomes and the cation-PEG-lipid-modified liposomes were obtained. The diameter, polydispersity index (PDI) and zeta-potential (surface charge) of the treated liposomes were evaluated by dynamic light scattering using Zetasizer Nano ZS (Malvern Instruments Co., Ltd., Worcestershire, U.K).

Results

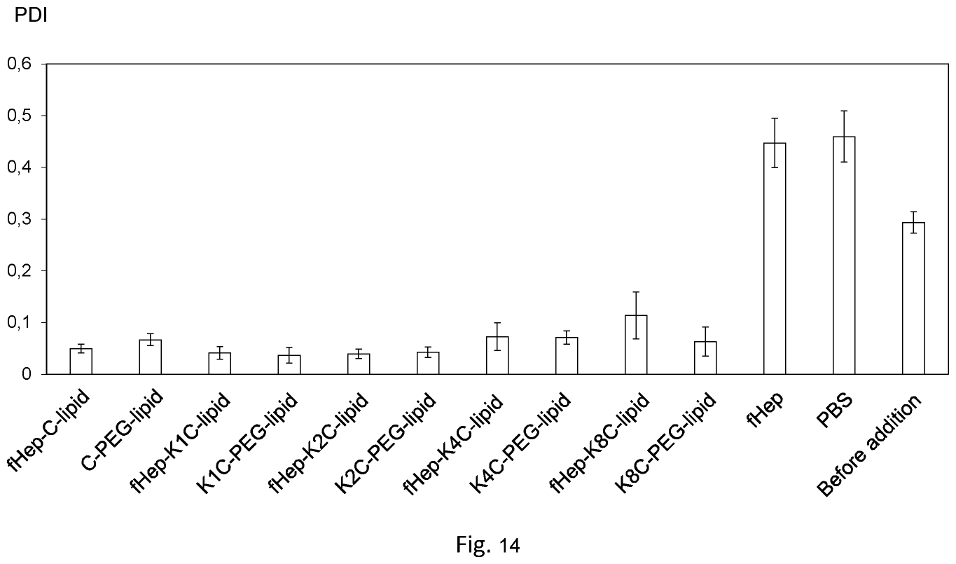

The size of liposomes, which were modified with fHep-lipid or cation-PEG-lipid, was measured by DLS (FIG. 13). As control groups, liposomes were treated with either PBS or fHep. Before the treatment, the size of liposome was 150 nm. Then, the average size of liposomes modified with fHep-lipid or cation-PEG-lipid was between 155 nm and 175 nm, while the size of control liposomes was approximately 250 nm. Also, the polydispersity index (PDI) of liposomes modified with fHep-lipid or cation-PEG-lipid showed lower value (-0.2) than that of control liposomes (-0.5) (FIG. 14). These results showed that liposomes modified with fHep-lipid or cation-PEG-lipid dispersed well, while the control liposomes were aggregated.

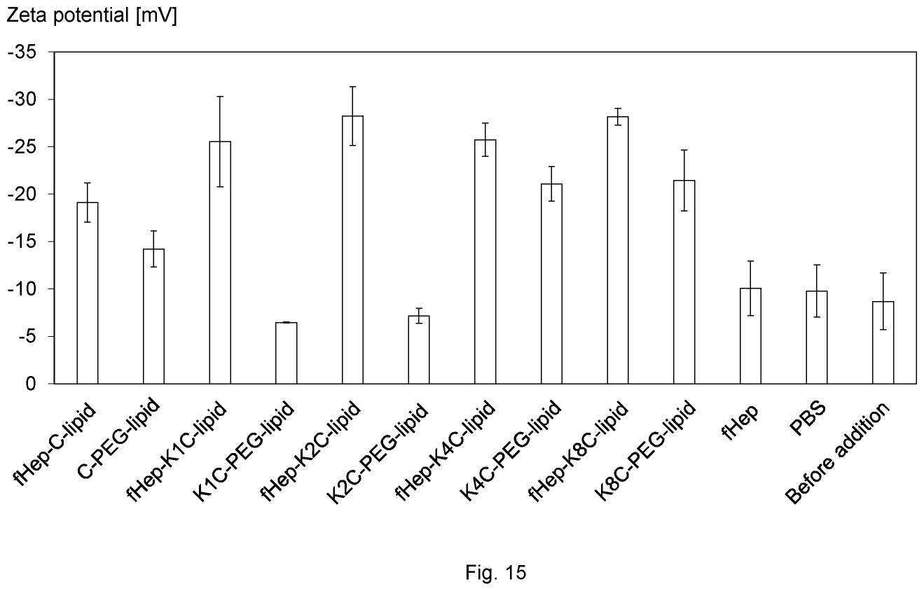

Also, the zeta potential of all liposomes was measured (FIG. 15). All samples showed negative charge, but liposomes modified with each fHep-lipid showed more negative charge than liposomes modified with each cation-PEG-lipid. This result showed that liposome surface was modified with negative fHep-lipid.

### Example 6—Cell Surface Functionalization with fHep(−)-Lipid

Human red blood cells (RBCs) were collected from a healthy donor using vacuum blood collection tube. Labelling of antithrombin (AT) was performed according to the protocol provided by the company using Alexa fluor™ 488 Antibody labeling kit. RBCs (10 μL, 7×10⁹ cells/mL in 10 mM EDTA/PBS) were rinsed with 1 mL PBS and centrifuged (Force mini SBC 140-115, BM EQUIPMENT Co., LTD, 1 min). The cell pellet was treated with fHep(−)-lipid (fHep-C(−)-lipid, fHep-K1C(−)-lipid, fHep-K2C(−)-lipid, fHep-K4C(−)-lipid, and fHep-K8C(−)-lipid), K1C-PEG-lipid, (0.5 mg/mL, 20 μL for each sample), fHep (4 mg/mL in PBS) or PBS (20 μL) for 30 minutes at RT followed by twice rinse with 1 mL PBS. The cell pellet was treated with Alexa488-AT (4 mg/mL) for 10 min at RT followed by twice rinse with 1 mL PBS and centrifuge (Force mini SBC 140-115, 1 min.). The obtained cell pellet was suspended in 1 mL PBS. The treated cells were observed using confocal microscopy (CLSM, LSM880, Carl Zeiss, Jena, Germany), and the cells were analyzed by flow cytometry (BD LSR II, BD Biosciences, San Jose, Calif., USA). The experiments were approved by ethical committee of The University of Tokyo.

The function of fHep-lipid was evaluated by FXa activity assay. Here we evaluated the binding capacity of antithrombin (AT) against each fHep-lipid, which was incorporated into living cells (CCRF-CEM cells). In order to modify the cell surface of CCRF-CEM cells, fHep-lipid (fHep-C-lipid) was mixed with the cells. The cell suspension (2×10⁶ cells in 2 mL RPMI 1640 medium) was washed with PBS by centrifugation (120 g, 4° C., 3 min) twice. A solution of fHep-C-lipid (100 μL, 0.5 mg/mL, in PBS containing 1 mg/mL glycine) was mixed with the cell pellet and incubated at RT for 30 min with gentle tapping every 10 min. As a control, fHep (100 μL, 2.5 mg/mL, in PBS containing 1 mg/mL glycine) was used. Then, treated cells were washed with PBS by centrifugation (180 g, 4° C., 6 min) twice. Finally, the cells were suspended in PBS (100 μL). The cell viability and cell number were evaluated using trypan blue and cell counter.

Then, the cell suspension (15 μL) was prepared and then mixed with human AT (15 μL) in a 96 well-plate. Bovine FXa (75 μL) was added into each well and incubated at RT for 120 sec. Then, after coloring reagent (75 μL) was mixed for 90 sec, citric acid aqueous solution (100 μL, 20 mg/mL) was added. Finally, the absorbance (at 405 nm) was measured.

Results

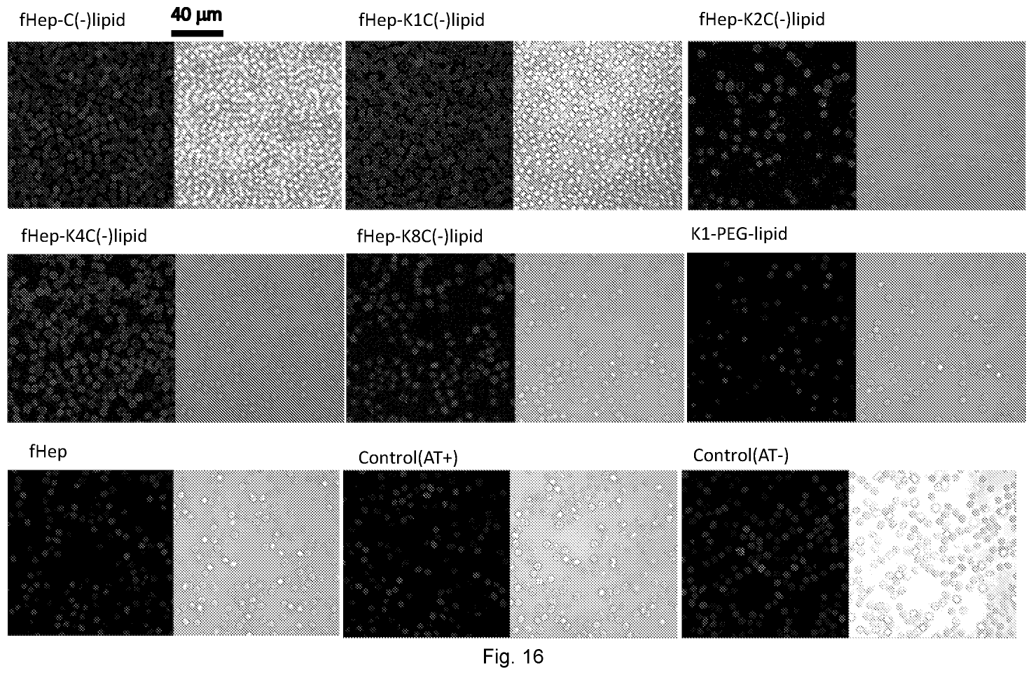

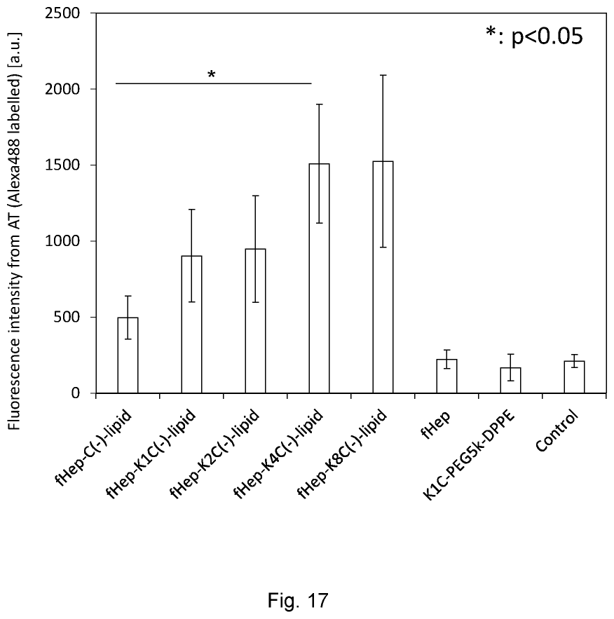

Fluorescence was observed on the cell membrane when cells were treated with fHep(−)-lipids (FIG. 16) whereas no fluorescence was observed on the cellular membrane when the cells were treated with K1C-PEG-lipid, fHep and PBS, indicating that AT is specifically immobilized onto fHep(−)-lipids on the cell surface. FIG. 17 showed the quantitative analysis of immobilized Alexa488-AT onto each cell, which also indicated that AT is specifically immobilized onto fHep(−)-lipids on the cell surface. In addition, the number of immobilized AT was highest when the cells were treated with fHep-K4C(−)-lipid and fHep-K8C(−)-lipid where highly packed fHep could effectively immobilize AT on fHep(−)-lipids.

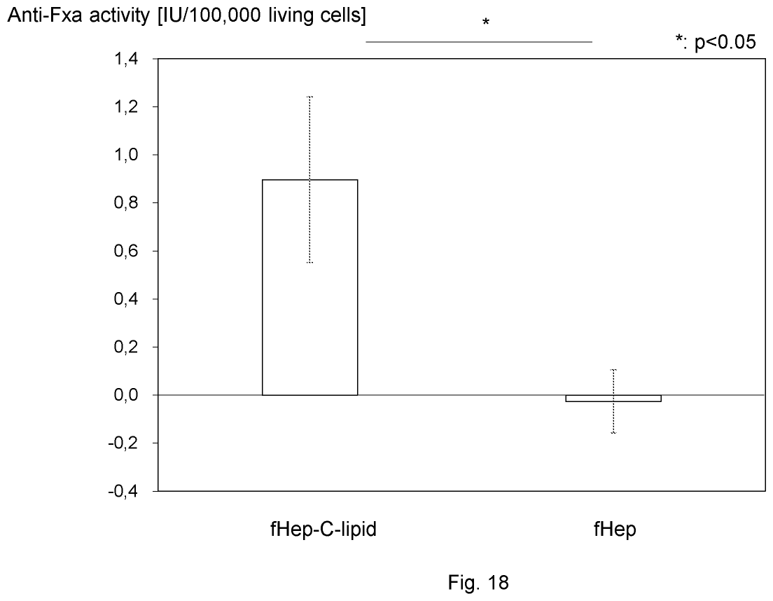

Anti-FXa activity of fHep-lipid modified cells (CCRF-CEM cells) was evaluated (FIG. 18). As control groups, non-modified cells were used for the assay. The anti-FXa activity was compensated by the cell number. fHep-lipid modified cells showed higher anti-FXa activity than non-modified cells (FIG. 18). These results showed that the surface of cells can be modified with fHep-lipid and also show that such surface modification results in anti FXa activity.

### Example 7—Functional Evaluation of fHep-Lipid Using Whole Blood Model

hMSCs Surface Functionalization with fHep-Lipid

hMSCs were cultured with DMEM (supplemented with 10% FBS, 50 IU/mL Penicillin, 50 μg/mL Streptomycin) at 37° C. in 5% CO₂ and 95% air. hMSCs (1 mL, 2.5x10⁵ cells/mL in PBS) collected by trypsinization (3 min, at 37° C., 5% CO₂) were centrifuged (Force mini SBC 140-115, BM EQUIPMENT Co., LTD, 1 min). The cell pellet was treated with fHep(−)-lipid (20 μL, 10 mg/mL in PBS, fHep-K1C(−)-lipid and fHep-K8C(−)-lipid), KₙC-PEG-lipid (20 μL, 10 mg/mL in PBS, K1C-PEG-lipid and K8C-PEG-lipid), fHep(20 μL, 30 and 120 mg/mL in PBS) or PBS (20 μL) for 30 min at RT, followed by twice rinse with cold PBS (1 mL) and centrifuge (Force mini SBC 140-115, 1 min). The samples that included fHep (fHep-K1C(−)-lipid, fHep-K8C(−)-lipid and fHep (30 or 120 mg/mL)) were reacted with glycine (18 mg/mL in PBS) for 4 hr, followed by purification with spin column to inactivate cytotoxic aldehyde group of free fHep in the solution. The cell pellet was treated with Alexa488-AT (4 mg/mL) for 10 min at RT, followed by once rinse with 1 mL cold PBS and centrifuge (Force mini SBC 140-115, 1 min.). The obtained cell pellet was suspended in 500 μL PBS, and the viability of the cells was evaluated using trypan blue and cell counter (countess II, Invitrogen). Those treated cells were observed using CLSM (LSM880, Carl Zeiss), and also the cells were analyzed by flowcytometry (BD LSR II, BD Biosciences).

Blood Test Using Human Whole Blood

hMSCs were exposed to human whole blood using chandler loop model [6] to evaluate the antithrombogenic property of the surface of the hMSCs treated with fHep-lipid. The passage number of hMSCs used for blood test was 6-8. hMSCs (1 mL, 1.0×10⁶ cells/mL in PBS) were treated with fHep-K1C(−)-lipid, fHep-K8C(−)-lipid and K1C-PEG-lipid (40 μL, 10 mg/mL in PBS, for each sample) and rinsed twice to remove free fHep-lipid. The viability and concentration of the cells were evaluated using trypan blue and cell counter (countess II, Invitrogen), and the cell concentration was adjusted at 2.5×10⁶ or 2.5x10⁵ cells/mL. The loop, made of polyurethane tube (ϕ6.3 mm, 40 cm) and polypropylene connector (ϕ6.5 mm, ISIS Co., Ltd., Osaka, Japan), was coated with MPC polymer (2 mL, 5 mg/mL in EtOH) for 24 hr, followed by drying in air for 24 hr to prevent surface-induced blood activation. Human whole blood was drawn into vacuum tube (7 mL, non-treated, TERUMO Corporation) from healthy donor who had received no meditation at least 14 days before blood donation. Immediately after blood collection, UFH (2.5 μL/1 mL blood, 200 IU/mL in PBS) was mixed to the blood. Then, human whole blood (2.5 mL, with 0.5 IU/mL UFH) was added into the MPC polymer-coated loop, followed by the addition of 100 μL of hMSCs suspension in PBS (2.5×10⁶ or 2.5×10⁵ cells/mL, treated or non-treated hMSCs) or PBS as a control. The tubes were rotated at 22 rpm for 2 hr in 37° C. cabinet. The blood collection (1 mL) from each loop was performed at 1 and 2 hr, and mixed with EDTA solution (10 mM). The platelets count was measured for each sample using cell counter (pocH-80i, SYSMEX, Hyogo, Japan). Then, the blood samples were centrifuged (TOMY MX301, 2,600 g, 15 min, 4° C.), and the plasma for each sample was collected and preserved in −80° C. freezer for enzyme linked immune-sorbent assay (ELISA) for TAT, C3a and sC5b-9. The experiments were approved by ethical committee of The University of Tokyo.

Measurement of TAT, C3a and sC5b-9 in Plasma

TAT, C3a and sC5b-9 in plasma was measured by conventional sandwich ELISA. Briefly, plasma was diluted with dilution buffer (PBS containing 0.05% TWEEN® 20, 10 mM EDTA and 10 mg/mL BSA). C3a in plasma was captured by anti-human C3a mAb 4SD17.3 which is precoated on 96-well plate and detected by a biotinylated polyclonal rabbit anti-C3a antibody and horse radish peroxidase (HRP)-conjugated streptavidin. TMB was reacted with fixed HRP (15 min), and the reaction was stopped with 1 M H₂SO₄ aq. Finally, the absorbance at 450 nm was detected using plate reader (AD200, Beckman Coulter, Miami, Fla., USA). Zymosan activated serum, calibrated against purified C3a, was used as a standard. The ELISA for sC5b-9 was demonstrated in the same way as C3a measurement. First, plasma was diluted with dilution buffer. Then, sC5b-9 in the plasma was captured by anti-neoC9 mAb aE11 (Diatec Monoclonals AS, Oslo, Norway), which was precoated on 96-well plate and detected with anti-human C5 polyclonal rabbit antibody (Dako) and HRP-conjugated anti-rabbit IgG (Dako). TMB was reacted with fixed HRP (15 min), and the reaction was stopped with 1 M H₂SO₄ aq, followed by the measurement of the absorbance at 450 nm using plate reader. Zymosan activated serum was used as a standard.

TAT was measured by an ELISA kit (Human Thrombin-Antithrombin Complex (TAT) AssayMax ELISA Kit, Assaypro, St Charles, Mo., USA) according to the company's instruction. Briefly, plasma was diluted with a diluent. Then, TAT was captured by a monoclonal antibody against human antithrombin which is precoated on 96-well plate and detected with biotinylated polyclonal antibody against human thrombin, and then, HRP-conjugated streptavidin. Peroxidase chromogen substrate, tetramethylbenzidine was reacted for 20 min, and reaction was stopped with 0.5 N hydrochloric acid solution, followed by the measurement of the absorbance at 450 nm using plate reader. Human TAT complex was used as a standard.

Results

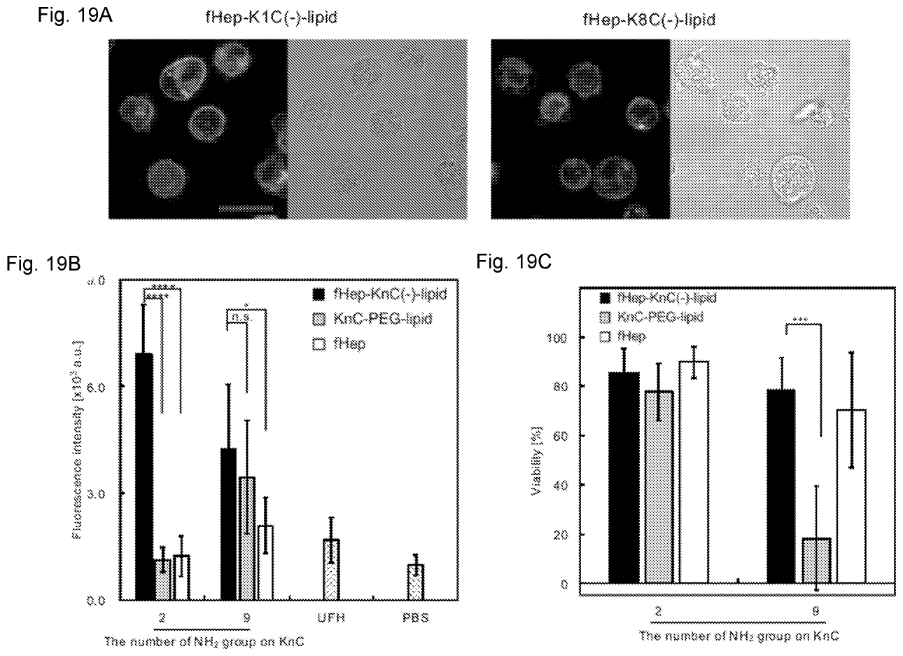

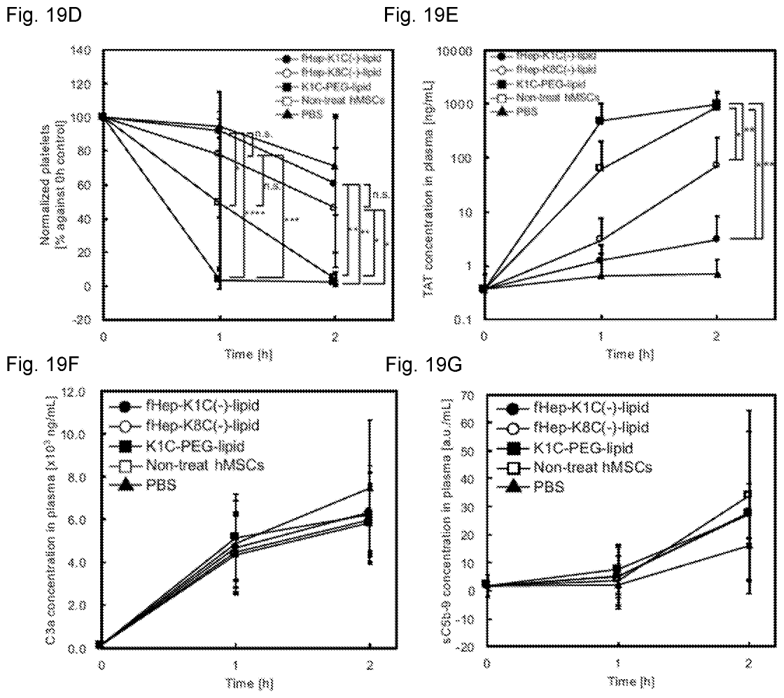

The surface of hMSCs was modified with fHep-lipids with higher and lower AT-binding ability, fHep-K1C(−)-lipid and fHep-K8C(−)-lipid, to compare the antithrombogenic property in human whole blood. The strong fluorescence from Alexa488-AT on the hMSCs membrane was observed when hMSCs were treated with fHep-K1C(−)-lipid and fHep-K8C(−)-lipid, whereas no fluorescence was observed on the cellular membrane when those cells were treated with KₙC-PEG-lipid (n=1 and 8), fHep and PBS (FIG. 19A), indicating that fHep(−)-lipids is immobilized on the hMSCs surface. The flow cytometry analysis showed that fHep-K1C-lipid-treated hMSCs had more binding of Alexa488-AT than fHep-K8C-lipid-treated hMSC did (FIG. 19B). This was probably because AT-binding was inhibited onto fHep-K8C(−)-lipid on hMSC surface due to the highly packed fHep of fHep-K8C(−)-lipid on hMSC surface, where exogeneous AT could not fully access to fHep. The viability of treated hMSCs was approximately 80%, which was similar to the control groups (UFH-treated, PBS-treated and non-treated cells), indicating the non-cytotoxicity of fHep(−)-lipids modification (FIG. 19C). High fluorescence intensity was observed for K8C-PEG-lipid-treated cells (FIGS. 19B, 19C). It was found that those cells were destroyed by K8C-PEG-lipid modification due to the cationic property, resulting in the uptake of Alexa488-AT.

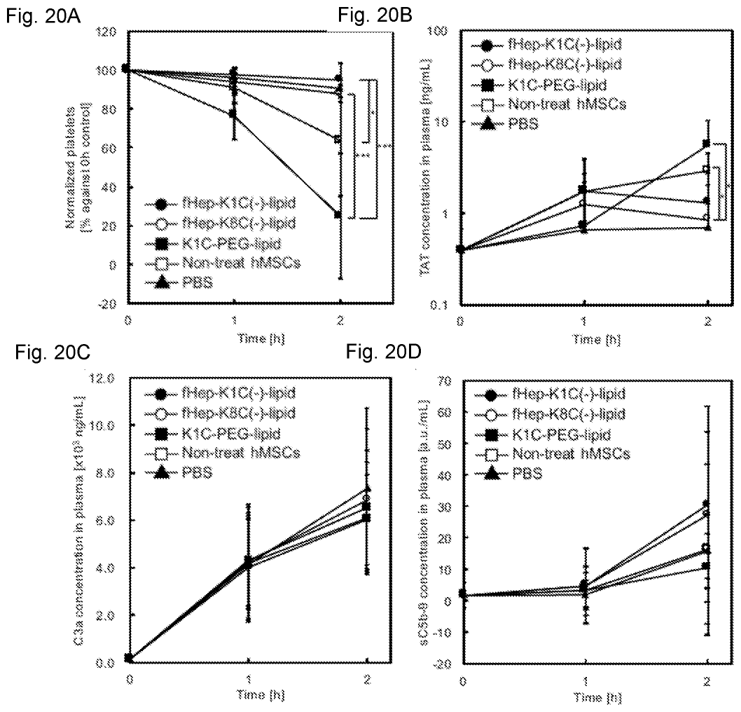

Next, we incubated human whole blood with hMSCs, which were treated with fHep-K1C(−)-lipid, fHep-K8C(−)-lipid or K1C-PEG-lipid with the concentration of 1.0×10⁴ (FIGS. 20A-20D) or 1.0×10⁵ cells/mL (FIGS. 19D-19G). Here non-treated hMSC and PBS were used as a control.

FIG. 19D shows the platelets count in the blood at 1 and 2 hr. There was almost no platelets reduction when we added PBS into the blood. Also, the platelet count reduced with time when we added hMSC, indicating that TF from hMSCs induced platelets aggregation. The same result was observed for K1C-PEG-lipid-modified hMSCs. It seems that the positively charged K1C at the end of PEG layer induced the platelet activation, so that platelet actually aggregated. On the other hand, in the case of hMSCs treated with fHep-K1C(−)-lipid and fHep-K8C(−)-lipid, although the platelets count slightly decreased, the remaining platelet was much higher than the control group of non-modified hMSCs, indicating that surface modification with fHep-lipid could attenuate platelet activation. There was no clear difference in platelet count between fHep-K1C(−)-lipid and fHep-K8C(−)-lipid-modified hMSCs. When the hMSC concentration was 1.0×10⁴ cells/mL, there was no clear difference in platelet count although we could see the similar tendency as seen in the higher cell concentration (FIG. 20A)

We evaluated the level of TAT, a coagulation marker during the 2 h incubation with treated hMSCs ([hMSC]=1.0×10⁴ cells/mL for FIG. 20B and 1.0×10⁵ cells/mL for FIG. 19E). There was a large increase in the TAT level with time for hMSCs modified with K1C-PEG-lipid and non-treated hMSCs, whereas there was a slight increase for hMSCs modified with fHep-lipid as well as PBS-added blood. This result was well pronounced in the blood of 1.0×10⁵ cells/mL hMSC. There were significant differences between non-treated hMSC group or K1C-PEG-lipid modified hMSCs and each fHep-lipid modified hMSCs (FIG. 19E). No significant differences between PBS mixed blood and each fHep-lipid modified hMSCs-mixed blood were seen. These results indicated that the fHep-lipid on hMSCs surface was able to suppress the coagulation activation.

In addition, we evaluated the generation of C3a and sC5b-9, complement markers during the 2 h incubation with treated hMSCs ([hMSC]=1.0×10⁴ cells/mL for FIGS. 20C, 20D and 1.0×10⁵ cells/mL for FIGS. 19F, 19G). Basically, the level of both markers increased with time. However, there was no difference in the level among the groups. We could not see any influence on the complement activation by the cell surface modification with fHep-lipid.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

### REFERENCES

1 Cabric S, Eich T, Sanchez J, Nilsson B, Korsgren O, Larsson L. A new method for incorporating function al heparin onto the surface of islets of Langerhans. Tissue Engineering, Part C, 14: 141-147. 2008.
2 U.S. Pat. No. 8,153,147
3 U.S. Pat. No. 9,795,629
4 Teramura Y, Oommen O P, Olerud J, Hilborn J, Nilsson B. Microencapsulation of cells, including islets, within stable ultra-thin membranes of maleimide-conjugated PEG-lipid with multifunctional crosslinkers. Biomaterials 34: 2683-2693. 2013.
5 Teramura, Y.; Kuroyama, K.; Takai, M. Influence of Molecular Weight of Peg Chain on Interaction between Streptavidin and Biotin-Peg-Conjugated Phospholipids Studied with Qcm-D. Acta Biomater 2016; 30, 135-143.
6 Asif, S.; Ekdahl, K. N.; Fromell, K.; Gustafson, E.; Barbu, A.; Le Blanc, K.; Nilsson, B.; Teramura, Y. Heparinization of Cell Surfaces with Short Peptide-Conjugated Peg-Lipid Regulates Thromboinflammation in Transplantation of Human Mscs and Hepatocytes. Acta Biomater 2016; 35, 194-205.
7 Luan, N. M.; Teramura, Y.; Iwata, H. Layer-by-Layer Co-Immobilization of Soluble Complement Receptor 1 and Heparin on Islets. Biomaterials 2011; 32, 6487-6492.
8 Cabric, S.; Sanchez, J.; Lundgren, T.; Foss, A.; Felldin, M.; Kallen, R.; Salmela, K.; Tibell, A.; Tufveson, G.; Larsson, R.; Korsgren, O.; Nilsson, B. Islet Surface Heparinization Prevents the Instant Blood-Mediated Inflammatory Reaction in Islet Transplantation. Diabetes 2007; 56, 2008-2015.
9 Kristina N Ekdahl, Shan Huang, Bo Nilsson, Yuji Teramura, Complement inhibition in biomaterial—and biosurface-induced thromboinflammation, Semin Immunol, 2016; 28(3): 268-77. doi: 10.1016/j.smim.2016.04.006.

1.-27. (canceled)

28. A method of producing a poly(ethylene glycol) lipid (PEG-lipid) comprising:
mixing a cation-PEG-lipid comprising at least one amino group with a sulfated glycosaminoglycan comprising at least one carbonyl group, preferably at least one aldehyde group, to form a Schiff base intermediate; and
adding a reducing agent to the Schiff base intermediate to form a sulfated glycosaminoglycan-PEG-lipid.

29. The method according to claim 28, further comprising mixing a maleimide-conjugated PEG-lipid with KₙC and/or CKₙ to form the cation-PEG-lipid comprising at least one amino group, wherein C is cysteine, K is lysine and n is zero or a positive integer equal to or smaller than 20.

30. The method according to claim 29, wherein n is zero or a positive integer equal to or smaller than 10.

31. The method according to claim 29, further comprising:
mixing α-N-hydroxysuccinimidyl-ω-maleimidyl PEG (NHS-PEG-Mal), triethylamine and 1,2-dipalmitoyl-sn-glycerol-3-phosphatidylethanolamine (DPPE) in dicholoromethane; and
precipitating the maleimide-conjugated PEG-lipid by adding diethyl ether to the mixture of NHS-PEG-Mal, triethylamine and DPPE in dicholormethane.

32. The method according to claim 28, wherein the sulfated glycosaminoglycan is fragmented heparin comprising at least one carbonyl group.

33. The method according to claim 32, wherein the sulfated glycosaminoglycan is fragmented heparin comprising at least one aldehyde group.

34. The method according to claim 32, further comprising:
mixing an acidic solution and a sodium nitrite (NaNO₂) aqueous solution to form a mixed solution;
adjusting the pH of the mixed solution within an interval of from 2 up to 6;
adding heparin to the mixed solution to form a heparin solution; and
adjusting the pH of the heparin solution within an interval of from 6 to 8 to form the fragmented heparin comprising at least one carbonyl group.

35. The method according to claim 34, further comprising:
dialyzing the fragmented heparin comprising at least one carbonyl group against water and lyophilizing the fragmented heparin comprising at least one carbonyl group

36. The method according to claim 34, wherein
adjusting the pH of the mixed solution comprises adjusting the pH of the mixed solution within an interval of from 3 up to 5;
adding heparin comprises adding heparin sodium to the mixed solution to form the heparin solution; and
adjusting the pH of the heparin solution comprises adjusting the pH of the heparin solution within an interval of from 6.5 to 7.5 to form the fragmented heparin comprising at least one carbonyl group;

37. The method according to claim 28, wherein adding the reducing agent comprises adding sodium cyanoboronhydride to the Schiff base intermediate to form the sulfated glycosaminoglycan-PEG-lipid.

38. The method according to claim 28, further comprising converting any unreacted amino groups in the sulfated glycosaminoglycan-PEG-lipid into carboxylic groups.

39. The method according to claim 38, further comprising adding an anhydride to the sulfated glycosaminoglycan-PEG-lipid to convert any unreacted amino groups in the sulfated glycosaminoglycan-PEG-lipid into carboxylic groups.

40. A poly(ethylene glycol) lipid (PEG-lipid) comprising at least one sulfated glycosaminoglycan attached to the PEG-lipid via a bond formed between an amino group of a cation-PEG-lipid comprising at least one amino group and a carbonyl group of the at least one sulfated glycosaminoglycan comprising at least one carbonyl group to form a Schiff base intermediate that is reduced by addition of a reducing agent.

41. The PEG-lipid according to claim 40, wherein the PEG-lipid comprises at least one sulfated glycosaminoglycan attached to the PEG-lipid via a bond formed between the amino group of the cation-PEG-lipid comprising at least one amino group and an aldehyde group of the at least one sulfated glycosaminoglycan comprising at least one aldehyde group to form the Schiff base intermediate that is reduced by addition of the reducing agent.

42. The PEG-lipid according to claim 40, wherein the PEG-lipid comprises a KₙC and/or CKₙ link interconnecting the at least one sulfated glycosaminoglycan and the PEG-lipid, wherein C is cysteine, K is lysine and n is zero or a positive integer equal to or smaller than 20.

43. The PEG-lipid according to claim 42, wherein n is selected within the interval of from 0 to 10.

44. The PEG-lipid according to claim 42, wherein the sulfated glycosaminoglycan is attached to the PEG-lipid via a bond formed between an amino group of any lysine residue in the KₙC and/or CKₙ link or an N-terminal amine in the KₙC and/or CKₙ link and a carbonyl group of the at least one sulfated glycosaminoglycan comprising at least one carbonyl group.

45. The PEG-lipid according to claim 44, wherein the sulfated glycosaminoglycan is attached to the PEG-lipid via a bond formed between the amino group of any lysine residue in the KₙC and/or CKₙ link or the N-terminal amine in the KₙC and/or CKₙ link and an aldehyde group of the at least one sulfated glycosaminoglycan comprising at least one aldehyde group.

46. The PEG-lipid according to claim 40, wherein the sulfated glycosaminoglycan is fragmented heparin.

47. The PEG-lipid according to claim 46, wherein the fragmented heparin has a weight average molecular weight (M_{w}) selected within the interval of from 2.5 kDa to 15 kDa.

48. The PEG-lipid according to claim 47, wherein the fragmented heparin has a M_{w} selected within the interval of from 5 kDa to 10 kDa.

49. The PEG-lipid according to claim 40, wherein any free amino groups in the sulfated glycosaminoglycan-PEG-lipid are converted into carboxylic groups.

50. The PEG-lipid according to claim 40, wherein the PEG-lipid has affinity for antithrombin and Factor H.

51. A biological tissue comprising at least one poly(ethylene glycol) lipid (PEG-lipid) according to claim 40, wherein the at least one PEG-lipid is anchored in cell membrane of the biological tissue.

52. The biological tissue according to claim 51, wherein the biological tissue is selected from the group consisting of islets of Langerhans, mesenchymal stem cells (MSCs), embryonic stem cells (ESCs), endothelial cells, beta cells, erythrocytes, hepatocytes, kidney, heart, pancreas, liver, lung, uterus, urinary bladder, thymus, intestine and spleen.

53. A liposome comprising at least one poly(ethylene glycol) lipid (PEG-lipid) according to claim 40, wherein the at least one PEG-lipid is anchored in a lipid bilayer of the liposome.

54. An in vitro method of providing biological tissue with a sulfated glycosaminoglycan coating, the in vitro method comprising adding in vitro poly(ethylene glycol) lipids (PEG-lipids) according to claim 40 to the biological tissue to anchor the PEG-lipids in cell membranes of the biological tissue.

55. An ex vivo method of treating an organ or a part of the organ, the method comprising:
ex vivo infusing a solution comprising poly(ethylene glycol) lipids (PEG-lipids) according to claim 40 into a vascular system of the organ or the part of the organ; and
ex vivo incubating the solution comprising the PEG-lipids in the vascular system to enable coating of at least a portion of the endothelial lining of the vascular system with the PEG-lipids.

56. The ex vivo method according to claim 55, wherein ex vivo incubating comprises ex vivo incubating the solution comprising the PEG-lipids in the vascular system to enable coating of at least a portion of the endothelial lining of the vascular system with the PEG-lipids while keeping the organ or the part of the organ submerged in an organ preservation solution comprising the PEG-lipids.

57. A method for treating, inhibiting or preventing a disease selected from the group consisting of thromboinflammation, instant blood-mediated inflammatory reaction (IBMIR), ischemia reperfusion injury (IRI), stroke and/or myocardial infarction in a subject, the method comprises administering poly(ethylene glycol) lipids (PEG-lipids) according to claim 40 to a subject in need thereof.
